(19) United States
(12) Patent Application Publication
(10) Pub. No.: US 2021/0215692 A1
(43) Pub. Date: Jul. 15, 2021

(54) COMPOSITIONS AND METHODS FOR TREATING INFLAMMATORY DISEASES

(71) Applicant: University of Washington, Seattle, WA (US)

(72) Inventors: Jan Christian Lood, Seattle, WA (US); Keith Elkon, Seattle, WA (US)

(73) Assignee: University of Washington, Seattle, WA (US)

(21) Appl. No.: 17/251,104

(22) PCT Filed: Jun. 10, 2019
(86) PCT No.: PCT/US2019/036398

§ 371 Date: Dec. 10, 2020

Related U.S. Application Data
(60) Provisional application No. 62/683,547, filed on Jun. 11, 2018.
(60) Provisional application No. 62/781,890, filed on Dec. 19, 2018.

(51) Int. Cl. G01N 33/564; C12N 15/113

(52) U.S. Cl. CPC G01N 33/564; C12N 15/1138; G01N 2800/102; G01N 2800/104; G01N 2333/70535; G01N 2333/908; G01N 2333/96441

(57) ABSTRACT

This disclosure relates to methods and composition for assessing conditions related to immune complex (IC)-mediated neutrophil activation and interventions to address the conditions. The disclosed methods include detecting the presence of ICs in a biological sample, and/or detecting the formation of neutrophil extracellular traps (NETs) in a biological sample. Other disclosed methods include detecting the modification or cleavage of FcgRIIA on circulating cells obtained from a patient. The assays and related compositions can identify patients with a severe phenotype and have the capacity to predict future disease flare and disease progression allowing for early preventive treatment and monitoring. The disclosure also provides compositions and kits to support performance of the disclosed methods.

### CROSS-REFERENCES TO RELATED APPLICATIONS

This application claims the benefit of U.S. Provisional Application No. 62/683,547, filed Jun. 11, 2018, and of U.S. Provisional Application No. 62/781,890, filed Dec. 19, 2018, the entire contents of which are incorporated herein by reference.

### STATEMENT REGARDING SEQUENCE LISTING

The sequence listing associated with this application is provided in text format in lieu of a paper copy and is hereby incorporated by reference into the specification. The name of the text file containing the sequence listing is UWOTL169528_ST25. The text file is 5 KB; was created on Jun. 10, 2019; and is being submitted via EFS-Web with the filing of the specification.

### BACKGROUND

Effective medical intervention for inflammatory and autoimmune diseases requires accurate diagnosis, characterization and monitoring. However, such accurate diagnostic tools have remained elusive for many conditions. For example, the diagnosis of lupus, even by certified rheumatologists, is difficult due to the heterogeneity of the disease, leading to errors in therapy, with concomitant side effects.

Circulating immune complexes (IC) are detectable in a variety of systemic diseases, including rheumatic and autoimmune diseases, as well as infectious diseases. Detection of circulating ICs can provide useful clinical information regarding underlying mechanisms contributing to disease, prognosis, treatment opportunities and monitoring of disease activity. There are a variety of tests that can detect ICs. The ones most commonly used in clinical laboratories are based on binding to C1q, detection of C3 fragments within the ICs, and/or precipitation with polyethylene glycol. However, in head-to-head studies, the overall agreement between the assays is about 50%. Given the inconsistency, and lack of reproducibility for many of the assays, the World Health Organization has recommended use of at least two test systems with different binding technology (e.g. antibody binding vs. complement binding) for clinical use. Main concerns relate to the ability of autoantibodies to interfere with the assay, e.g. anti-C1q antibodies binding to C1q, thus hindering recognition of C1q to the ICs, as well as rheumatoid factor (RF) binding to human IgG blocking their binding to the ELISA, and/or giving false positive test. Given the inconsistency and uncertainty to what is being measured, IC analyses are no longer routinely analyzed at all clinical laboratories.

Furthermore, ICs are heterogeneous and can have different effects on immune responses, thus leading to different manifestations of inflammatory and/or autoimmune conditions.

Though complement opsonization of IC is an important event in clearance of IC, complement opsonization leads to loss of inflammatory properties of the ICs, through complement receptor-mediated signaling. Thus, assessing complement-bearing ICs will primarily analyze the non-inflammatory ICs, and not the harmful inflammatory ICs. The inflammatory trigger instead relies on the ability of ICs to engage FcgRs on immune cells through the Fc portion of the IgG molecule. Some of the current ELISA kits address this aspect, including the C1q-binding assay. However, these assays, as discussed above, are flawed by presence of anti-C1q antibodies and rheumatoid factor in many of the patient samples.

Not all ICs share the same capacity to activate immune cells. We have demonstrated that ICs containing nucleic acids, e.g. DNA and RNA, have a high capacity to activate immune cells to induce inflammation. In neutrophils, nucleic acid-containing ICs lead to induction of a neutrophil cell death process termed NETosis, with extrusion of nuclear debris mixed with cytosolic and granular components in the form of neutrophil extracellular traps (NETs). This process, downstream of IC activation, has been shown to partake in inflammation and autoimmunity. However, there is currently no assay that considers circulating NETs in clinical diagnostics.

Thus, despite the advances in the understanding of inflammatory and autoimmune diseases, there remains a great need for sensitive and accurate detection assays to detect and characterize the status of such diseases, including prediction of the disease activity or flares, to support precise medical intervention. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure provides methods and compositions for detection, monitoring, and/or treating conditions characterized by aberrant inflammation and autoimmunity dysfunction.

In one aspect the disclosure provides a method of detecting the presence of immune complexes (ICs) in a biological sample obtained from a subject. The method comprises: contacting a biological sample with one or more particles expressing FcgRIIA receptor, or an extracellular domain thereof, on the surface of the particle;

contacting the biological sample with one or more affinity reagents that compete with ICs for binding an extracellular domain of FcgRIIA receptor on the one or more particles;

and detecting the binding of the one or more affinity reagents to one or more particles in the biological sample.

Reduced binding levels of the one or more affinity reagents compared to a reference binding level indicates the presence of elevated levels of ICs in the subject.

In some embodiments, the method further comprises detecting the presence of neutrophil extracellular traps (NETs) in a biological sample obtained from the subject. This detection step can comprise:

contacting the biological sample with a capture affinity reagent that binds to the NET at a first epitope;

contacting the biological sample with a detection affinity reagent that binds to the NET at a second epitope; and

detecting the binding of the detection affinity reagent to a captured NET.

Detected binding of the detectably labeled affinity reagent to the captured NET indicates the presence of NETs in the biological sample. An indicated presence of NETs in the biological sample in combination with detection of the elevated levels of ICs in the subject indicates the subject has or is at elevated risk of having an inflammatory or autoimmune disease.

In another aspect, the disclosure provides a method of determining the status of an autoimmune or inflammatory disease in a subject. The method comprises:

detecting a level of neutrophil extracellular traps (NETs) in a biological sample obtained from the subject; and

detecting a level of immune complexes (ICs) in the subject.

The combination of a higher level of NETs compared to a NET reference level and a higher level of ICs compared to an IC reference level indicate the presence or elevated risk of an autoimmune or inflammatory disease in the subject.

In yet another aspect, the disclosure provides a method of detecting circulating cells with a truncated FcgRIIA receptor. The method comprises:

contacting a sample containing one or more neutrophils and/or monocytes obtained from a subject with a first affinity reagent that specifically binds to a first epitope in an N terminal domain of the FcgRIIA receptor and a second affinity reagent that specifically binds to a second epitope in an extracellular domain of the FcgRIIA that is not in the N terminal domain; and

detecting the binding of the first affinity reagent and the second affinity reagent to the one or more neutrophils and/or monocytes in the sample.

Reduced binding levels of the first affinity reagent compared to the second affinity reagent indicate one or more neutrophils and/or monocytes with truncated FcgRIIA receptor. Elevated levels of neutrophils and/or monocytes with truncated FcgRIIA receptors indicate presence or increased risk of inflammatory or autoimmune disease.

In any aspect relating to detection, the disclosure further provides methods of treating a subject determined to have an inflammatory or autoimmune disease.

In yet another aspect, the disclosure provides a method of increasing phagocytosis of nucleic acid-containing immune complexes (ICs) by neutrophils. The method comprises contacting the neutrophils with an agent that inhibits activity of TLR7, TLR8 and/or TLR9.

In yet another aspect, the disclosure provides a method of reducing nucleic acid-containing immune complex (IC)-driven inflammation in a subject in need thereof, comprising administering to the subject an effective amount of a TLR7-9 inhibitory deoxynucleotide (iODN) that inhibits activity of TLR7, TLR8 and/or TLR9.

In yet another aspect, the disclosure provides a kit comprising affinity reagents described herein.

In one aspect, the kit can comprise a particle expressing FcgRIIA receptor, or an extracellular domain thereof, and one or more affinity reagents that compete with ICs for binding the extracellular domain of FcgRIIA receptor expressed on the particle.

In one aspect, the kit can comprise a capture affinity reagent that binds to a neutrophil extracellular trap (NET) at a first epitope, and a detection affinity reagent that binds to the NET at a second epitope.

In one aspect, the kit can comprise a first affinity reagent that specifically binds to a first epitope in an N-terminal domain of the FcgRIIA receptor; and a second affinity reagent that specifically binds to a second epitope in an extracellular domain of the FcgRIIA that is not in the N-terminal domain.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic overview of the role of FcgRIIA in neutrophil NETosis. Left panel: Neutrophils may commit to phagocytosis or NETosis based on environmental triggers, in particular TLR activation. Right panel: Depiction of key signaling events resulting in TLR-mediated regulation of IC-mediated inflammation by neutrophils, monocytes and pDCs. In brief, TLR activation results in activation of PI3K, contributing to generation of reactive oxygen species (ROS) via NADPH oxidase. ROS is essential for NET formation but also release of proteases able to shed FcgRIIA from immune cells. Loss of FcgRIIA results in increased ability of neutrophils to undergo IC-mediated NETosis, while also impairing phagocytic ability in neutrophils, monocytes and pDCs. Non-cleared ICs will instead activate the complement system to generate the anaphylatoxin, C5a, and be cleared through complement-dependent pathways.

FIGS. 2A-2D provide an overview of the IC-FLOW assay. 2A and 2B are schematics of the assay in absence (2A) and presence (2B) of ICs. 2C is a representative flow cytometry plot for IV.3 staining in absence or presence of IC, with a third indicated line (“no staining”) representing absence of detection antibody. D is a standard curve created by increasing amounts of heat-aggregated IgG ICs.

FIG. 3 graphically represents increased levels of ICs in SLE patients. Levels of ICs were measured by IC-FLOW technology and depicted as ug/mL using FUN-2 as reporting antibody.

FIGS. 4A-4C graphically illustrate that IC levels are associated with disease activity in SLE. Levels of ICs were analyzed by IC-FLOW technology and associated with clinical and immunological features of SLE including (4A) complement consumption (C+), (4B) presence of anti-dsDNA antibodies, and (4C) active lupus nephritis.

FIGS. 5A and 5B graphically illustrate that RA patients have circulating ICs. In 5A levels of circulating ICs were measured by IC-FLOW in RA patients. In 5B levels of circulating ICs were measured by IC-FLOW related to disease activity and number of swollen joints.

FIGS. 6A and 6B graphically illustrate that IC-FLOW can predict disease progression in RA. Levels of ICs were analyzed at baseline in a RA inception cohort (n=250) and assessed for associations with future (6A) joint space narrowing and (6B) erosion.

FIGS. 7A-7C illustrate an overview of the NET-ELISA method. 7A is a schematic illustrating an embodiment of the NET-ELISA assay as a sandwich ELISA using anti-MPO as a capture antibody and HRP-conjugated anti-dsDNA antibody as detection antibody. Bovine serum albumin (BSA) is used to block non-specific interactions. Only complexes containing both MPO and DNA (e.g. NETs) are detected. 7B is a representative picture of NETs used to establish a standard curve for the assay in 7C.

FIG. 8 graphically illustrates levels of NETs in SLE patients. NETs, assessed by NET-ELISA, were elevated in three distinct SLE cohorts (UW, CVD and act) as compared to healthy individuals (HC).

FIGS. 9A-9C graphically illustrates that NET-ELISA identifies a severe disease phenotype in SLE. 9A illustrates that patients with history of nephritis had elevated levels of NETs. Patients with high levels of NETs had increased flare frequency (9B) and concomitant increased average SLEDAI score (9C).

FIG. 10 graphically illustrates the predictive value of NET-ELISA for SLE flare in patients. Using a cohort of 60 SLE patients at time-point of remission, NET-ELISA predicted which patients were to develop a flare within three months.

FIGS. 11A and 11B graphically illustrate that NET-ELISA can identify patients with calcinosis in JDM, a pediatric rheumatic disease. 11A shows NET-ELISA levels in healthy children (HC), juvenile SLE, as well as pediatric myositis patients. 11B shows NET-ELISA levels in JDM children with calcinosis versus without calcinosis.

FIGS. 12A and 12B illustrates that calcium crystals can induce NETs. Human neutrophils were incubated with calcium crystals and assessed for NET formation using microscope (12A) and NET-ELISA (12B).

FIG. 13 graphically illustrates levels of NETs in RA patients. Levels of NETs were analyzed in three cross-sectional cohorts of RA patients, the latter one (RA3) being serum samples.

FIG. 14 graphically illustrates that NET levels are associated with disease activity in RA. Levels of NETs are increased in RA patients, even in remission, and associated with disease flare.

FIG. 15 graphically illustrates the combined risk score of NET-ELISA, IC-FLOW and CRP in evaluating disease activity in RA. A risk score, composed of NET-ELISA, IC-FLOW and CRP), was calculated and disease activity (CDAI) assessed in the different risk score groups.

FIGS. 16A and 16B graphically illustrate a comparison on IC levels using commercial assay (Quidel) (16A) or the IC-FLOW assay (16B).

FIGS. 17A-17I graphically illustrate IC levels in active disease stratified by indicated disease manifestations.

### DETAILED DESCRIPTION

The present disclosure provides compositions and improved methods for detection, characterization, and monitoring inflammatory and autoimmune diseases, such as manifestations of systemic lupus erythematosus (SLE) and rheumatoid arthritis (RA). The disclosed methods and compositions can be incorporated into treatment strategies to address such conditions in a more accurate and precise approach.

The disclosure is based on the inventors' work in characterizing the underlying mechanisms of neutrophil activation. As described in more detail below, the inventors demonstrated that FcgRIIA is the main FcgR responsible for uptake of IC by circulating immune cells, such as neutrophils and monocytes. TLR7/8 activation shifts neutrophils from phagocytosis of immune complexes (ICs) via the FcgRIIA receptor to NETosis. This activation shift to NETosis with reduced phagocytosis of immune complexes is associated with partial proteolytic cleavage of FcgRIIA. Cleaved FcgRIIA was found in SLE neutrophils ex vivo and thus established as a determinative marker for the activation of NETosis and, thus, the inflammatory condition in the SLE subjects. Given the difficulty to accurately quantify inflammatory ICs by standard ELISA techniques, the inventors designed a method to assess the activation status of immune cells by assaying the levels of truncated FcgRIIA. The method was demonstrated using flow cytometry, but could be applied using fluorescence microscopy, ImageStream, fluorimetry, or any other appropriate technique that is routinely practiced in the art that is based on imaging colored/labeled cells.

Also described below is a different approach to assaying immune cell activation and pro-inflammatory signaling leading to disease conditions that addresses IC binding to FcgRIIA. Again, the technique (referred to as IC-FLOW) was established using flow cytometry, but could be readily implemented using fluorescence microscopy, ImageStream, fluorimetry, or any other appropriate technique that is routinely practiced in the art that is based on imaging colored/labeled cells/particles. This technique has a particular advantage in that it avoids current caveats with circulating autoantibodies, yet specifically assays inflammatory ICs, e.g. ICs capable of engaging FcgRIIA. Yet another strategy is to directly assay NETs resulting from the NETosis activation pathway (referred to as NET-ELISA). This assay incorporates the dual recognition of two elements of NETs, e.g. a protein component, such as myeloperoxidase (MPO), neutrophil elastase (NE) and/or citrullinated histones, and DNA. An important benefit of this assay is the dual recognition of two components of NETs, increasing the specificity of the assay. While detection of NETs, or NETosis in general, is not necessarily specific to IC-mediated inflammation, such detection can supplement other assays as described herein directed to IC detection to detect and monitor IC-mediated inflammation and related conditions such as SLE and RA. Coordinated use of IC-FLOW and NET-ELISA can provide information on two key inflammatory components in inflammatory and autoimmune diseases. When combined they confer unique opportunity to assess the collected ‘risk’ of IC-mediated NET formation occurring in patients and permit characterization of the state and progression of diseases such as RA and in SLE.

In accordance with the foregoing, the disclosure provides several methods, and related compositions and kits, for detection of inflammatory and autoimmune diseases. The disclosed methods and related compositions can be integrated into methods of medical intervention. Various aspects of the disclosure will be addressed in turn.

### FcgRIIA Modification

As described in more detail below, the inventors discovered that neutrophil TLR7/8 activation shifts neutrophils from phagocytosis of immune complexes (ICs) to NETosis, a programmed necrosis pathway. Accordingly, the ICs remain in circulation and can induce higher incidence of inflammation.

Accordingly, in one aspect, the disclosure provides a method of increasing phagocytosis of nucleic acid-containing immune complexes (ICs) by neutrophils. The method comprises contacting the neutrophils with an agent that inhibits activity of TLR7, TLR8 and/or TLR9.

As used herein, the term immune complex (IC) refers to a complex of antibody and antigen that circulate through the body. In some embodiments and aspects, ICs contain nucleic acid molecules.

In one embodiment, the agent is a TLR7-9 inhibitory deoxynucleotide (iODN). iODNs are short nucleotide sequences able to interfere with the Toll-like receptors (TLR) that hinder the TLRs (e.g., TLR7, TLR8 and/or TLR9) binding to cognate ligands. Exemplary, non-limiting examples of iODNs are described in more detail in Barrat, F. J., et al., 2005. Journal of Experimental Medicine, 202(8):1131-1139, incorporated herein by reference in its entirety. Additional exemplary examples of iODNs are set forth in SEQ ID NOS:2-9. Persons of ordinary skill in the art can identify additional iODN species to inhibit activity of TLR7, TLR8 and/or TLR9 receptors on neutrophils.

In other embodiments, the agent inhibits endosomal acidification, such as hydroxychloroquine and salts thereof. Endosomal acidification is essential for the presentation of ligand to TLRs and, thus, prevention of endosomal acidification can inhibit activity of TLR7, TLR8, and/or TLR9. Additional agents that inhibit endosomal acidification are known.

In some embodiments, the increase in phagocytosis of ICs is associated with a reduced rate of programmed neutrophil necrosis (NETosis). The reduced rate can be determined by assaying subsequent neutrophil activity or presence of neutrophil extracellular traps (NETs), as described below in more detail.

In some embodiments, the method is performed in vitro or ex vivo to a subject from whom the neutrophils have been obtained.

In other embodiments, the neutrophils are contacted in vivo in a subject in need thereof, wherein an effective amount of the agent is administered to the subject. Accordingly, the disclosure also provides a method of reducing nucleic acid-containing IC driven inflammation in a subject in need thereof. In one embodiment, the method comprising administering to the subject an effective amount of a TLR7-9 inhibitory deoxynucleotide (iODN) that inhibits activity of TLR7, TLR8 and/or TLR9. In other embodiments, the method comprises administering an effective amount of an agent that inhibits endosomal acidification, such as hydroxychloroquine and salts thereof. Additional agents that inhibit endosomal acidification are known.

The agent or agents can be formulated appropriately for methods of treatment and administration for in vivo therapeutic settings in subjects (e.g., mammalian subjects with IC-driven inflammation, e.g., rheumatic inflammation, e.g., lupus) according to routine methods and knowledge in the art. For example, the disclosed agents can be formulated with appropriate carriers and non-active binders, and the like, for administration. Proper dosing can be routinely established.

In some embodiments, the subject in need of intervention for IC-driven inflammation has an autoimmune condition. In some embodiments, the autoimmune condition comprises rheumatic inflammation. In some embodiments, the autoimmune condition is systemic lupus erythematosus (SLE). In other embodiments, the autoimmune condition is rheumatoid arthritis (RA).

In another aspect, the disclosure provides a method of detecting circulating cells with a truncated FcgRIIA receptor. The method comprises:

contacting a sample containing one or more circulating cells obtained from a subject with a first affinity reagent that specifically binds to a first epitope in an N terminal domain of the FcgRIIA receptor and a second affinity reagent that specifically binds to a second epitope in an extracellular domain of the FcgRIIA that is not in the N terminal domain; and

detecting the binding of the first affinity reagent and the second affinity reagent to the one or more circulating cells in the sample.

A detection of reduced binding levels of the first affinity reagent compared to the second affinity reagent indicate one or more circulating cells with truncated FcgRIIA receptor.

As used herein, the term circulating cells refer to cells or cellular structures that circulate in the liquid systems of the body, such as in the blood, lymph, saliva, spinal fluid, and the like. The circulating cells can comprise immune cells, such as neutrophils and/or monocytes. The term circulating cells can also encompass platelets.

The term affinity reagent is defined in more detail below. In some embodiments, the first affinity reagent and the second affinity reagent are independently an antibody, or a fragment or a derivative thereof that retains antigen binding domain(s) of the source antibody.

In some embodiments, the first and affinity reagent is labeled with a first detectable label and the second affinity reagents is labeled with a second detectable label, wherein the first detectable label and the second detectable label are different. The different labels can emit signals that can be differentiated by routine techniques. For example, the different labels can emit different light at different wavelengths resulting different colors. The art is replete with available labels, such as fluorescent labels, that are routinely used for labeling molecules such as antibodies and which are encompassed by the present disclosure.

The FcgRIIA receptor is a receptor expressed on the surface of many circulating cells, such as neutrophils and monocytes. An exemplary amino acid sequence for human FcgRIIA receptor is disclosed as GenBank Accession No. P12318, incorporated herein by reference. The amino acid sequence is also set forth herein as SEQ ID NO:1 and is used herein for reference. It will be understood that reference to amino acids or amino acid positions that “correspond” to SEQ ID NO:1 refer to the same or homologous positions in relation to this reference sequence and allows for minor sequence variation, typically conservative variation that does not alter the identity of the protein as an FcgRIIA receptor.

The inventors have shown that the signaling pathway leading to inflammatory phenotypes, and away from phagocytosis of circulating ICs, involves induced proteolytic cleavage of the N-terminal portion of the extracellular domain of the FcgRIIA on circulating cells. The first affinity reagent specifically binds to a first epitope in an N-terminal domain of the FcgRIIA receptor that will be intact and associated with expressed FcgRIIA receptor in the absence of any induced cleavage, but in contrast will be cleaved and disassociated from the remainder of the expressed FcgRIIA receptor once cleavage has occurred. In some embodiments, the “N-terminal domain” comprises an amino acid sequence from the N-terminus of the FcgRIIA receptor to an amino acid that is N terminal to an amino acid corresponding to amino acid position 132 of SEQ ID NO:1. In some embodiments, the N-terminal domain comprises an amino acid sequence corresponding to amino acids 132-137 of SEQ ID NO:1. In some embodiments, the first epitope to which the first affinity reagent binds comprises amino acids that correspond to amino acids 132-137 of SEQ ID NO:1. Exemplary antibodies encompassed by this disclosure that bind to such an N-terminal domain include antibody IV.3 or antibody 8.7, see, e.g., Sardjono, C. T, et al., 2008, Epitope Mapping of Fc gamma RIIa Monoclonal Antibodies. Indonesian Journal of Biotechnology, 13(1):1030-1037; Ramsland, P. A., et al., 2012, J Immunol, 187(6):3208-3217, each of which is incorporated herein by reference in its entirety. Thus, in some embodiments, the first affinity reagent is or comprises antibody IV.3 or antibody 8.7. In related embodiments, the first affinity reagent is or comprises or an antigen binding fragment or derivative of antibody IV.3 or antibody 8.7.

The second epitope to which the second affinity reagent specifically binds is disposed in the extracellular domain of the FcgRIIA receptor with the caveat that it is not disposed in the N-terminal domain that is cleaved away upon the IC-induced signaling. In some embodiments, the second epitope is C-terminal to position 131 (i.e., closer to the C-terminus than position 131) but N-terminal to the transmembrane domain (i.e., closer to the N-terminus than the transmembrane domain). As the transmembrane domain is predicted to be from amino acid positions corresponding to positions 218-240 of SEQ ID NO:1, the second epitope will typically comprise amino acids within the sequence corresponding to amino acid positions 132 and 217 of SEQ ID NO:1. An exemplary antibody encompassed by this disclosure that binds to such an extracellular domain is antibody FUN-2. In some embodiments, the second affinity reagent is or comprises an antigen binding fragment or derivative that comprises the antigen binding domains of the FUN-2 antibody.

In some embodiments, the circulating cells are obtained from the subject. The circulating cells can be processed, cleaned, isolated, etc., and then placed in an appropriate liquid medium for the assay to provide the sample that is contacted. In other embodiments, the sample is a biological sample obtained from the subject. The biological sample can be or comprise blood, serum, plasma, synovial fluid, bronchial alveolar lavage (BAL), spinal fluid, saliva, or any bodily fluid that is likely to contain circulating cells, such as immune cells (e.g., neutrophils and/or monocytes).

As indicated, the method comprises detecting the binding of the first affinity reagent and the second affinity reagent to the one or more circulating cells (e.g., neutrophils, monocytes, and/or platelets) in the sample. The detection can be carried out in any acceptable assay format that can differentiate and quantify the detectable labels in the sample. For example, in some embodiments, the binding of the first affinity reagent and binding of the second affinity reagent are detected with flow cytometry, fluorescence microscopy, ImageStream, fluorimetry, or any other appropriate technique that is routinely practiced in the art that is based on imaging colored/labeled cells/particles. The binding of the second affinity reagent is an indicator of the total level of FcgRIIA receptor on the cells of the sample. The binding of the first affinity reagent is an indicator of the levels of proportion of the FcgRIIA receptors that are intact, i.e., not proteolytically cleaved due to IC-mediated signaling. Thus, an indicated presence of one or more circulating cells with truncated FcgRIIA receptor indicated by a reduced binding levels of the first affinity reagent compared to the second affinity reagent in the sample indicates the subject has active IC-mediated signaling to promote inflammation. In some embodiments, subjects that provide a sample with circulating cells expressing truncated (i.e., cleaved) FcgRIIA receptor have an autoimmune disease characterized by IC-mediated inflammatory signaling.

In some embodiments, the method further comprises determining a ratio of binding by the first affinity reagent to binding by the second affinity reagent in the sample. This experimental ratio is compared to a reference ratio. The reference ratio is a ratio of binding by the first affinity reagent to binding by the second affinity reagent in a reference sample obtained from one or more individuals that do not have an autoimmune disease. A low ratio of binding by the first affinity reagent to binding of the second affinity reagent in the sample compared to the reference ratio indicates (e.g., is further confirmation that) the subject has an immunological disease.

In some embodiments, the autoimmune disease is systemic lupus erythematosus (SLE). In some embodiments, the autoimmune disease is the autoimmune condition is rheumatoid arthritis (RA).

This aspect of the disclosure also provides a method of treating a subject determined to have an autoimmune or inflammatory condition. The autoimmune or inflammatory condition is typically characterized by IC-mediated inflammation. The term “treating” is defined in more detail below. Thus, upon determination of the presence of circulating cells in a subject with a truncated FcgRIIA receptor, the disclosed method can further comprise treating the subject for the autoimmune disease. Appropriate treatments for autoimmune diseases, such as SLE and RA are known and are encompassed by this disclosure. For example, in some approaches, agents are administered that block aspects of the immune system. For example, B cell depletion therapy can be used to lower the production of autoantibodies. Exemplary agents include Hydroxychloroquine (Plaquenil), which is commonly used and thought to affect TLR7/8 activation. Belimumab (Benlysta), is an antibody used for targeting B cells (the origin of autoantibodies), and thus reduce initiation of immune complexes. Rituximab (Rituxan), is an antibody used for B cell depletion therapy to reduce autoantibodies and immune complex levels. In other embodiments, steroids or other anti-inflammatory agents can be used for appropriate treatment. Prednisone is an exemplary steroid used as a general anti-inflammation to suppress ongoing disease.

The subject of this aspect can be any animal that can suffer from autoimmune disease. In some embodiments, the subject is a human or non-human mammal, such as another primate, horse, dog, mouse, rat, guinea pig, rabbit, and the like.

In another aspect, the disclosure provides a kit that comprises a first affinity reagent that specifically binds to a first epitope in an N-terminal domain of the FcgRIIA receptor, and a second affinity reagent that specifically binds to a second epitope in an extracellular domain of the FcgRIIA that is not in the N-terminal domain.

The first affinity reagent and the second affinity reagent can independently be an antibody, or a fragment or a derivative thereof, as defined herein. The first and affinity reagent can be labeled with a first detectable label and the second affinity reagents can be labeled with a second detectable label, wherein the first detectable label and the second detectable label are different.

The affinity reagents are defined in more detail above. In some embodiments, the first epitope to which the first affinity reagent binds is or comprises amino acids corresponding to amino acids 132-137 of SEQ ID NO:1. In some embodiments, the first affinity reagent is or comprises antibody IV.3 or antibody 8.7, or an antigen binding fragment or derivative thereof. In some embodiments, the second epitope to which the second affinity reagent binds is disposed between amino acids corresponding to amino acid positions 132 and 217 of SEQ ID NO:1. In some embodiments, the second affinity reagent is or comprises antibody FUN-2, or an antigen binding fragment or derivative thereof.

The kit can comprise written indicia instructing how to obtain the sample, how to contact the sample with the affinity reagents, and/or how to detect binding. The kit can also comprise reference standards or ratio values reflecting binding of the affinity reagents to circulating cells from healthy subjects.

### IC-FLOW

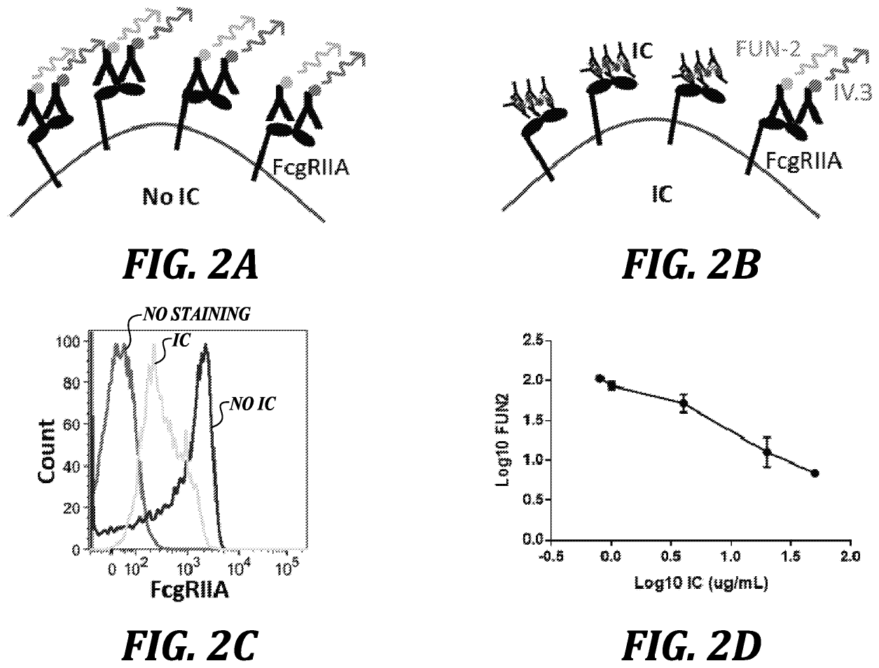

As indicated herein, not all ICs cause inflammation, which makes direct quantification of total circulating ICs problematic when assessing inflammatory-related conditions. Thus, alternative approaches for ascertaining inflammatory ICs have developed. As inflammatory ICs bind to the extracellular domain of FcgRIIA receptor, the IC-FLOW assay disclosed herein is directed to determining the availability of this extracellular domain after exposure to fluids that potentially contain inflammatory ICs. The indicated availability of the extracellular domain for binding by an affinity reagent is inversely proportional to the presence of inflammatory ICs in the sample. See, e.g., FIGS. 2A and 2B.

Accordingly, in another aspect the disclosure provides a method of detecting the presence of immune complexes (ICs) in a biological sample obtained from a subject. The method comprises: contacting a biological sample with one or more particles expressing FcgRIIA receptor on the surface; contacting the biological sample with one or more affinity reagents that compete with ICs for binding an extracellular domain of FcgRIIA receptor on the one or more particles; and detecting the binding of the one or more affinity reagents to one or more particles in the biological sample. Reduced binding levels of the one or more affinity reagents compared to a reference binding level indicates the presence of elevated levels of ICs in the subject.

The one or more particles that express FcgRIIA receptor on the surface can be cell-based or synthetic particles. For example, cells can be provided that express natural, endogenous FcgRIIA receptor on the cell surface. Such cells can be neutrophils, monocytes, platelets, etc. Such cells can be sourced from one or more donor individuals that do not have elevated levels of inflammatory ICs and, thus, the provided cells express FcgRIIA with exposed, unbound extracellular domains on their surfaces. In some embodiments, the donor individual(s) is/are from the same species as the subject. In other embodiments, the cells can be any transgenic cell that has heterologous FcgRIIA receptor (or at least the extracellular domain thereof) expressed on the cell surface and cultured in the absence of inflammatory ICs. In yet other embodiments, the particles can be synthetic, non-cellular based particles such as cell, liposomes, mixed micelles, synthetic beads, solid nanoparticles, and the like, that have at least the extracellular domain of the FcgRIIA receptor tethered to the particle surface. An exemplary extracellular domain of the FcgRIIA receptor can correspond to a sequence from the N-terminus to about amino acid number 217 of SEQ ID NO:1.

In some embodiments, such as illustrated in FIGS. 2A and 2B, multiple affinity reagents that bind to distinct epitopes on the FcgRIIA receptor extracellular domain can be used. Without being bound by any particular theory, multiple affinity reagents can increase the sensitivity of the signal. In some embodiments, the method comprises contacting the sample with a first affinity reagent and a second affinity reagent. Each of the first affinity reagent and the second affinity reagent compete with ICs for binding the extracellular domain of FcgRIIA receptor. However, the first affinity reagent and the second affinity reagent do not mutually compete for binding the extracellular domain of FcgRIIA receptor to allow their simultaneous binding to available FcgRIIA receptor (i.e., not bound with ICs).

The one or more affinity reagents are typically detectably labeled, as described above with respect to detecting FcgRIIA receptor truncation. Thus, in some embodiments the method comprises contacting the sample with a first affinity reagent and a second affinity reagent, wherein the first and affinity reagent is labeled with a first detectable label and the second affinity reagents is labeled with a second detectable label, and wherein the first detectable label and the second detectable label are different.

The detection can be carried out in any acceptable assay format that can differentiate and quantify the detectable labels in the sample. For example, in some embodiments, the binding of the affinity reagents (e.g., binding of the first affinity reagent and binding of the second affinity reagent) are detected with flow cytometry, fluorescence microscopy, ImageStream, fluorimetry, or any other appropriate technique that is routinely practiced in the art that is based on imaging colored/labeled cells/particles.

This aspect of the disclosure encompasses any relevant affinity reagent, such as defined in more detail below. In some embodiments, the one or more affinity reagents are independently an antibody, or an antigen-binding fragment or a derivative thereof. An exemplary first affinity reagent is or comprises antibody IV.3 or antibody 8.7, or an antigen binding fragment or derivative of antibody IV.3 or antibody 8.7, as described in more detail above. An exemplary second affinity reagent is or comprises antibody FUN-2, or an antigen binding fragment or derivative thereof as described in more detail above.

In some embodiments, the biological sample from the subject comprises blood, serum, plasma, synovial fluid, bronchoalveolar lavage, spinal fluid, saliva, and the like including any bodily fluid that is likely to contain circulating ICs.

Considering that the one or more affinity reagents compete with ICs for binding to the extracellular domain of FcgRIIA receptor on the particles, a reduction in binding of the detectable affinity reagents is indicative of competition from the presence of ICs (see, e.g., FIG. 2B). Thus, to determine a reduction, a comparison can be made to a reference standard. In some embodiments, the reference binding level is a level of binding by the one or more affinity reagents to the extracellular domain of FcgRIIA in a reference sample with IC levels associated with one or more individuals with no inflammatory or autoimmune disease.

Multiple reference standards with known quantities of ICs can also be used according to persons of ordinary skill in the art to create a reference curve to quantify ICs in the sample obtained from the subject. In some embodiments, the indicated presence of elevated levels of ICs in the subject indicates the subject has or is at elevated risk of having an inflammatory or autoimmune disease. In some embodiments, an indication of elevated levels of ICs in the subject indicates the relative severity of inflammatory or autoimmune disease.

In some embodiments, the presence of elevated levels of ICs in the subject indicates the subject has systemic lupus erythematosus (SLE). In some embodiments, the presence of elevated levels of ICs in the subject indicates the subject has active SLE disease. Active SLE is a term used to refer disease activity that exceeds an SLEDAI (an index of activity) more than 4. In some embodiments, the presence of elevated levels of ICs in the subject indicates the subject has an elevated risk of a disease flare. A flare of SLE refers to a measurable worsening of the disease condition from one point to the next, e.g., between clinical assessments. A flare can be characterized in some instances according to threshold differences in activity, such as at least a change of 1, 2, 3, 4 or more on a SLEDAI scale between clinical visits. In some embodiments, the indication of risk of disease flare address the risk within a period of about 1, 2, 3, 4, 5, 6, 7, 8, 9, 10, 11, 12, 13, 14, or 15 weeks.

In other embodiments, the presence of elevated levels of ICs in the subject indicates the subject has rheumatoid arthritis (RA). In some embodiments, the presence of elevated levels of ICs in the subject indicates the subject has an elevated risk of developing erosive joint disease.

In yet other embodiments, the presence of elevated levels of ICs in the subject indicates the subject has juvenile dermatomyositis (JDM).

This aspect of the disclosure also provides a method of treating a subject determined to have an autoimmune or inflammatory condition. The autoimmune or inflammatory condition is characterized by IC-mediated inflammation. The term “treating” is defined in more detail below. Thus, upon determination of the presence the presence of immune complexes (ICs) in the biological sample obtained from a subject, the method can further comprise treating the subject for the autoimmune or inflammatory disease. All appropriate and treatments and interventions for inflammatory diseases such as SLE, RA, and JDM are contemplated in this disclosure. Exemplary compositions used for such interventions are described in more detail above.

The disclosed method can also include detection of other known biomarkers for autoimmune or inflammatory diseases, tested from the same or different biological samples from the subject. Exemplary additional biomarkers encompassed by the disclosure include ANA and anti-dsDNA antibodies for purposes of SLE diagnosis; anti-dsDNA antibodies, complement c3/c4 levels for SLE disease activity; anti-ACPA antibodies for RA diagnosis; and sedimentation rates and CRP for RA disease activity.

In additional embodiments, the method of detecting the presence of immune complexes (ICs) as described above also includes detecting the presence of neutrophil extracellular traps (NETs) in a biological sample obtained from the subject. Detection of NETs is described in more detail below and is also encompassed in this aspect of the application. Briefly, the element of detecting the presence of NETs in a biological sample obtained from the subject comprises:

contacting the biological sample with a capture affinity reagent that binds to the NET at a first epitope;

contacting the biological sample with a detection affinity reagent that binds to the NET at a second epitope; and

detecting the binding of the detection affinity reagent to a captured NET.

In these embodiments, the detected binding of the detectably labeled affinity reagent to the captured NET indicates the presence of NETs in the biological sample. An indicated presence of NETs in the biological sample in combination with detection of the elevated levels of ICs in the subject indicate the subject has or is at elevated risk of having an inflammatory or autoimmune disease.

In another aspect, the disclosure provides a kit that comprises a particle expressing FcgRIIA receptor, or an extracellular domain thereof, and one or more affinity reagents that compete with ICs for binding the extracellular domain of FcgRIIA receptor expressed on the particle, which are described above in more detail.

Briefly, in some embodiments the one or more affinity reagents that compete with ICs for binding an extracellular domain of FcgRIIA receptor on the particle expressing FcgRIIA receptor comprises a first affinity reagent and a second affinity reagent. The first affinity reagent and the second affinity reagent each compete with ICs for binding the extracellular domain of FcgRIIA receptor but wherein the first affinity reagent and the second affinity reagent do not mutually compete for binding the extracellular domain of FcgRIIA receptor. In some embodiments, the one or more affinity reagents are detectably labeled, as described above. The one or more affinity reagents can be independently an antibody, or a fragment or a derivative thereof. In some embodiments, the one or more affinity reagent are selected from antibody IV.3 or antibody 8.7 (e.g., as a first affinity reagent), FUN-2 (e.g., as a second affinity reagent), or an antigen binding fragment or derivative thereof.

In some embodiments, the kit also comprises a capture affinity reagent that binds to a neutrophil extracellular trap (NET) at a first epitope, and a detection affinity reagent that binds to the NET at a second epitope. The capture affinity reagent and the detection affinity reagent, the second detection affinity reagent are independently an antibody, or a fragment or a derivative thereof. The detection affinity reagent can be detectably labeled.

The kit can comprise written indicia instructing how to obtain the sample, how to contact the sample with the one or more particles, the one or more affinity reagents, and/or how to detect binding. The kit can also comprise reference standards or ratio values reflecting binding of the affinity reagents to circulating cells from healthy subjects.

### NET-ELISA

Neutrophil extracellular traps (NETs) are the result of a neutrophil cell death process in which DNA is extruded together with cytoplasmic and granular content to eliminate extracellular pathogens. NETs can be the result in the inflammatory signaling pathway for neutrophils and other immune cells. As described herein, the presence of NETs is associated with inflammation and autoimmune conditions.

Thus, in another aspect, the disclosure provides a method of detecting the presence of neutrophil extracellular traps (NETs) in a biological sample obtained from a subject. The method can be an element that is combined with other assays (such as IC-FLOW, described above) or can be performed alone to detect or monitor associated disease (such as during the course of treatment). The method comprises: contacting the biological sample with a capture affinity reagent that binds to the NET at a first epitope; contacting the biological sample with a detection affinity reagent that binds to the NET at a second epitope; and detecting the binding of the detection affinity reagent to captured NET. A detected binding of the detectably labeled affinity reagent to the captured NET indicates the presence of NETs in the biological sample.

In some embodiments, the capture affinity reagent is immobilized on a solid substrate, such as a well surface or a particle.

NETs typically comprise nucleic acids and a combination of certain proteins such as myeloperoxidase (MPO), neutrophil elastase (NE), and citrullinated histones. Accordingly, in some embodiments, the NET being detected minimally comprises a complex myeloperoxidase (MPO) and nucleic acid, a complex of neutrophil elastase (NE) and nucleic acid, and/or a complex of citrullinated histones and DNA. In some embodiments, the first epitope is on the MPO, NE, or citrullinated histone within the NET complex. The second epitope comprises double stranded DNA. Alternatively, it will be understood that the first epitope can comprise double stranded DNA whereas the second epitope is on the MPO, NE, or citrullinated histone on the NET complex.

An exemplary, non-limiting affinity reagent that binds to an epitope on MPO is an anti-human MPO antibody (Biorad, #0400-0002), which is encompassed in this disclosure. An exemplary, non-limiting affinity reagent that binds to DNA is an anti-dsDNA antibody (Roche, #11544675001). Other exemplary affinity reagents that bind to dsDNA are labeled dyes known to bind to the dsDNA, for example Sytox-Green, Pico-Green, and the like. Such dyes are encompassed by the disclosure as affinity reagents that bind to dsDNA epitope in a NET. An exemplary, non-limiting affinity reagent that binds to NE is an anti-neutrophil elastase antibody (Calbioshem, #481001).

In some embodiments, the detection affinity reagent is detectably labeled. Detectable labels, such as fluorescent labels are described above. Alternatively, a detectable label can be configured to emit a detectable signal upon action on a substrate, such as with horseradish peroxidase. Appropriate detectable labels are well-understood in the art and can be implemented into the disclosed method by persons of ordinary skill in the art.

In some embodiments, the method further comprises contacting the sample with a second detection affinity reagent that specifically binds to the detection affinity reagent. In such embodiments, the second detection reagent has a detectable label and serves to provide a detectable signal on the bound and immobilized NET.

In any embodiment, the capture affinity reagent, the first detection reagent, and/or the second detection affinity reagent can be independently an antibody, or a fragment or a derivative thereof, as described herein.

In some embodiments, the biological sample is selected from blood, serum, plasma, synovial fluid, bronchoalveolar lavage, spinal fluid, saliva and the like In some embodiments, the biological sample from the subject comprises blood, serum, plasma, synovial fluid, bronchoalveolar lavage, spinal fluid, saliva, and the like including any bodily fluid that is likely to contain circulating NETs.

In some embodiments, the indicated presence of NETs in the biological sample indicates the subject has circulating NETs and accordingly has or is at elevated risk of having an inflammatory or autoimmune disease. In some embodiments, an indication of elevated levels of NETs in the subject indicates the relative severity or activity of inflammatory or autoimmune disease. Elevated levels of NETs can be determined by comparing the detected level to reference standard levels. Such reference standard levels can be determined from samples obtained from one or more individuals without an inflammatory or autoimmune condition (e.g., from the same species as the subject) and/or from samples with known levels of NETs. In some embodiments, the known levels of the NETs are associated with disease indications, activities, or severity. The method can be incorporated into a method of monitoring an inflammatory disease state or condition over a period of time. In some embodiments, the period of time can include administration of therapeutic intervention for the disease or condition.

In some embodiments, the presence of elevated levels of NETs in the biological sample indicates the subject has systemic lupus erythematosus (SLE). In some embodiments, the indicated presence of NETs in the biological sample indicates the subject has increased risk of disease flare, nephritis, and/or myocardial infarction associated with SLE. In some embodiments, the indication of risk addresses the risk within a period of about 1, 2, 3, 4, 5, 6, 7, 8, 9, 10, 11, 12, 13, 14, or 15 weeks.

In some embodiments, the indicated presence of NETs in the biological sample indicates the subject has calcinosis associated with juvenile dermatomyositis (JDM).

In some embodiments, the indicated presence of NETs in the biological sample indicates the subject has rheumatoid arthritis (RA). In some embodiments, the indicated presence of NETs in the biological sample indicates the subject has increased risk of developing extra articular disease (EAD) associated with RA. The EAD can be, for example interstitial lung disease (ILD) or extra articular nodules.

This aspect of the disclosure also provides a method of treating a subject determined to have an autoimmune or inflammatory condition. The term “treating” is defined in more detail below. Thus, upon determination of the presence of NETs in the biological sample obtained from a subject, the method can further comprise treating the subject for the autoimmune or inflammatory disease. All appropriate and treatments and interventions for inflammatory diseases such as SLE, RA, and JDM are contemplated in this disclosure. Exemplary compositions used for such interventions are described in more detail above.

As indicated above, this method of detecting NETs can be combined with assays for other markers of inflammatory or autoimmune diseases, such as the IC-FLOW assay described above, which detects the presence of inflammatory ICs in the subject. This specific combination is described in more detail below.

In another aspect, the disclosure provides a kit that comprises a capture affinity reagent that binds to a neutrophil extracellular trap (NET) at a first epitope, and a detection affinity reagent that binds to the NET at a second epitope, which are described above in more detail.

In some embodiments, the kit further comprises a solid substrate. In some embodiments, the capture affinity reagent is immobilized on the solid substrate. In some embodiments, the first epitope to which the capture affinity reagent binds is on a myeloperoxidase (MPO), a neutrophil elastase (NE), or citrullinated histone on the NET complex and the second epitope comprises double stranded DNA. In other embodiments, the first epitope to which the capture affinity reagent binds comprises double stranded DNA and the second epitope is on a myeloperoxidase (MPO), a neutrophil elastase (NE), or citrullinated histone on the NET complex.

In some embodiments, the detection affinity reagent is detectably labeled. In other embodiments, the kit further comprises a second detection affinity reagent that specifically binds to the detection affinity reagent, wherein the second detection affinity reagent is detectably labeled.

In some embodiments, the capture reagent, the detection reagent, and the second capture reagent are independently selected from an antibody, or an antigen binding fragment or derivative thereof.

An exemplary, non-limiting affinity reagent that binds to an epitope on MPO encompassed by this aspect is an anti-human MPO antibody (Biorad, #0400-0002), which is encompassed in this disclosure. An exemplary, non-limiting affinity reagent that binds to DNA is an anti-dsDNA antibody (Roche, #11544675001).

In some embodiments, the kit further comprises a particle expressing FcgRIIA receptor, or an extracellular domain thereof, and one or more affinity reagents that compete with ICs for binding the extracellular domain of FcgRIIA receptor expressed on the particle. These and other components of the IC-FLOW-related kit described above in more detail are contemplated for this kit.

The kit can also comprise written indicia instructing how to obtain the sample, how to contact the sample with the capture and detection affinity reagents, and/or how to detect binding. The kit can also comprise reference standards reflecting various levels of NETs in reference individuals or with reference conditions

### Dual Detection

As indicated above, the method of biomarker detection described herein can be conducted alone or in combination with assays for other biomarkers. Often, combination of multiple markers for a condition can lead to more nuanced revelation of characteristics of conditions or diseases in a subject. For example, more precise distinction can be made regarding disease severity, activity, or specific risk thereof. As described below, the combination of the IC-FLOW assay with the NET-ELISA assay provided a synergistic effect to ascertain characteristics of autoimmune disease, including aspects of SLE and RA.

Accordingly, in another aspect the disclosure provides a method of determining the status of an autoimmune or inflammatory disease in a subject. The method comprises: detecting a level of neutrophil extracellular traps (NETs) in a biological sample obtained from the subject; detecting a level of immune complexes (ICs) in the subject. The combination of a higher level of NETs compared to a NET reference level and a higher level of ICs compared to an IC reference level indicate the presence or elevated risk of an autoimmune or inflammatory disease in the subject.

As described above, in one embodiment the step of detecting the NETs in the biological sample comprises: contacting the biological sample with a capture affinity reagent that specifically binds to the NET at a first epitope; contacting the biological sample with a detection affinity reagent that specifically binds to the NET at a second epitope; and detecting the binding of the detection affinity reagent to a captured NET. A detected binding of the detectably labeled affinity reagent to the captured NET indicates the presence of NETs in the biological sample.

Additional aspects of the step(s) of detecting NET in the sample and inferring the presence of NETs in the subject are described in more detail above and are encompassed by this aspect of the disclosure. Briefly, in some embodiments the capture affinity reagent is immobilized on a solid substrate. The NET can comprise a complex myeloperoxidase (MPO) and nucleic acid, a complex of neutrophil elastase (NE) and nucleic acid, and/or a complex of citrullinated histones and DNA. In some embodiments, the first epitope is on the MPO, NE, or citrullinated histone on the NET complex, and the second epitope comprises double stranded DNA. In some embodiments, the first epitope comprises double stranded DNA and the second epitope is on the MPO, the NE, or the citrullinated histone on the NET complex.

In some embodiments, the detection affinity reagent is detectably labeled. In some embodiments, the method further comprises contacting the sample with a second detection affinity reagent that specifically binds to the detection affinity reagent, wherein the second detection affinity reagent is detectably labeled.

In some embodiments, the biological sample from which NETs are assayed is selected from blood, serum, plasma, synovial fluid, bronchoalveolar lavage, spinal fluid, saliva, and the like including any bodily fluid that is likely to contain circulating NETs.

As described above, in one embodiment the step of detecting the ICs in the subject comprises: contacting a biological sample obtained from a subject with one or more particles expressing FcgRIIA receptor on the surface; contacting the biological sample with one or more affinity reagents that compete with ICs for binding an extracellular domain of FcgRIIA receptor on the one or more particles; and detecting the binding of the one or more affinity reagents to one or more particles in the biological sample. Reduced binding levels of the one or more affinity reagents compared to a reference binding level indicates the presence of elevated levels of ICs in the subject.

Additional aspects of the step(s) of detecting ICs in the sample and inferring the presence of NETs in the subject are described in more detail above and are encompassed by this aspect of the disclosure. Briefly, the step of detecting the ICs in the subject comprises contacting the sample with a first affinity reagent and a second affinity reagent, wherein the first affinity reagent and the second affinity reagent each compete with ICs for binding the extracellular domain of FcgRIIA receptor but wherein the first affinity reagent and the second affinity reagent do not mutually compete for binding the extracellular domain of FcgRIIA receptor. The one or more affinity reagents can be detectably labeled.

In some embodiments, detecting ICs in the subject can comprise contacting the sample with a first affinity reagent and a second affinity reagent, wherein the first and affinity reagent is labeled with a first detectable label and the second affinity reagents is labeled with a second detectable label, and wherein the first detectable label and the second detectable label are different.

In some embodiments, first affinity reagent is or comprises antibody IV.3 or antibody 8.7, or an antigen binding fragment or derivative thereof. In some embodiments, the second affinity reagent is or comprises antibody FUN-2, or an antigen binding fragment or derivative thereof. The one or more particles can comprise one or more of neutrophils, monocytes, liposomes, mixed micelles, platelets, synthetic beads, and the like. As described above, the cell-based particles can express endogenous or exogenous FcgRIIA receptor. In some embodiments, the expressed FcgRIIA receptor is full length or near full-length. In other embodiments, the cell expresses at least a portion of the extracellular domain. In other embodiments, the particle is a synthetic particle, such as a liposome, micelle, synthetic bead, solid nanoparticle, and the like. In such embodiments, the particle has at least a portion of the extracellular domain tethered thereto.

In some embodiments, the capture affinity reagent, the detection affinity reagent, the second detection affinity reagent, and/or the one or more affinity reagents are independently an antibody, or a fragment or a derivative thereof.

Detection of binding of the one or more affinity reagents to the one or more particles can be performed using flow cytometry, fluorescence microscopy, ImageStream, fluorimetry, or any other appropriate technique that is routinely practiced in the art that is based on imaging colored/labeled cells/particles.

In some embodiments, the sample contains wherein the biological sample from which ICs are assayed comprises blood, serum, plasma, synovial fluid, bronchoalveolar lavage, spinal fluid, saliva, and the like including any bodily fluid that is likely to contain circulating ICs.

In some embodiments, the biological sample from which the NETs are assayed is the same biological sample from which ICs are assayed. In other embodiments, the biological sample from which the NETs are assayed is a different biological sample from which ICs are assayed.

In some embodiments, the reference binding level is a level of binding by the one or more affinity reagents to the extracellular domain of FcgRIIA in a reference sample with IC levels associated with one or more individuals with no inflammatory or autoimmune disease.

In some embodiments, the autoimmune or inflammatory disease being detected is systemic lupus erythematosus (SLE), as described herein. For example, the indicated presence or elevated risk of an autoimmune or inflammatory disease in the subject comprises an indication that the subject with SLE has an increased risk of a flare. In other embodiments, the autoimmune condition is rheumatoid arthritis (RA), as described herein. For example, the indicated presence or elevated risk of an autoimmune or inflammatory disease in the subject comprises an indication that the subject with RA has an increased risk of a flare. In other embodiments, the autoimmune condition is juvenile dermatomyositis (JDM), as described herein. For example, the indicated presence or elevated risk of an autoimmune or inflammatory disease in the subject comprises an indication that the subject with JDM has an increased risk of calcinosis.

This aspect also provides a method of treating a subject determined to have an autoimmune or inflammatory disease. Thus, in some embodiments, the method further comprises administering a therapeutic agent to the subject to treat the autoimmune or inflammatory disease, as described in more detail above.

This aspect also provides a method of monitoring the status of the autoimmune or inflammatory disease in the subject over a period of time. The monitoring includes performing the described steps at multiple time points within a defined period of time to ascertain the status or character of the condition, e.g., whether the condition is stable, progressing, in remission, or changing to other indications, etc. In some embodiments, the defined period of time includes administration of a therapy or other intervention to the subject. The method can assist a care provider to understand the efficacy of the therapy or intervention.

In another aspect, the disclosure provides a kit that comprises:

a capture affinity reagent that binds to a neutrophil extracellular trap (NET) at a first epitope, and

a detection affinity reagent that binds to the NET at a second epitope; and a particle expressing FcgRIIA receptor, or an extracellular domain thereof, and one or more affinity reagents that compete with ICs for binding the extracellular domain of FcgRIIA receptor expressed on the particle.

The elements of the kit are a combination of kits that are described in more detail above with respect to IC-FLOW and NET-ELISA detection strategies.

### General Definitions

Unless specifically defined herein, all terms used herein have the same meaning as they would to one skilled in the art of the present disclosure. Practitioners are particularly directed to Ausubel, F. M., et al. (eds.), Current Protocols in Molecular Biology, John Wiley & Sons, New York (2010), Coligan, J. E., et al. (eds.), Current Protocols in Immunology, John Wiley & Sons, New York (2010), Mirzaei, H. and Carrasco, M. (eds.), Modern Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

For convenience, certain terms employed in this description and/or the claims are provided here. The definitions are provided to aid in describing particular embodiments and are not intended to limit the claimed invention, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense, which is to indicate, in the sense of “including, but not limited to.” Words using the singular or plural number also include the plural and singular number, respectively.

The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The term “affinity reagent” refers to any molecule having an ability to bind to a specific target molecule (i.e., antigen of interest and/or target antigen) with a specific affinity (i.e., detectable over background). Affinity reagent molecules are known and have been characterized for useful antigens and are encompassed by the present application without limitation. Exemplary and non-limiting categories of affinity reagents that can be used in the context of the present disclosure include antibodies, and antigen fragments and derivatives thereof.

The term “antibody” is used herein in the broadest sense and encompasses various antibody structures derived from any antibody-producing mammal (e.g., mouse, rat, rabbit, and primate including human), and which specifically bind to an antigen of interest. An antibody fragment specifically refers to an intact portion or subdomain of a source antibody that still retains antigen-biding capability. An antibody derivative refers to a molecule that incorporates one or more antibodies or antibody fragments. Typically there is at least some additional modification in the structure of the antibody or fragment thereof, or in the presentation or configuration of the antibody or fragment thereof. Exemplary antibodies of the disclosure include polyclonal, monoclonal and recombinant antibodies. Exemplary antibodies or antibody derivatives of the disclosure also include multispecific antibodies (e.g., bispecific antibodies); humanized antibodies; murine antibodies; chimeric, mouse-human, mouse-primate, primate-human monoclonal antibodies; and anti-idiotype antibodies.

As indicated, an antibody fragment is a portion or subdomain derived from or related to a full-length antibody, preferably including the complementarity-determining regions (CDRs), antigen binding regions, or variable regions thereof, and antibody derivatives refer to further structural modification or combinations in the resulting molecule. Illustrative examples of antibody fragments or derivatives encompassed by the present disclosure include Fab, Fab′, F(ab)₂, F(ab′)₂ and Fv fragments, diabodies, single-chain antibody molecules, V_{H}H fragments, V_{NAR} fragments, multispecific antibodies formed from antibody fragments, nanobodies and the like. For example, an exemplary single chain antibody derivative encompassed by the disclosure is a “single-chain Fv” or “scFv” antibody fragment, which comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Another exemplary single-chain antibody encompassed by the disclosure is a single-chain Fab fragment (scFab).

As indicated, antibodies can be further modified to created derivatives that suit various uses. For example, a “chimeric antibody” is a recombinant protein that contains domains from different sources. For example, the variable domains and complementarity-determining regions (CDRs) can be derived from a non-human species (e.g., rodent) antibody, while the remainder of the antibody molecule is derived from a human antibody. A “humanized antibody” is a chimeric antibody that comprises a minimal sequence that conforms to specific complementarity-determining regions derived from non-human immunoglobulin that is transplanted into a human antibody framework. Humanized antibodies are typically recombinant proteins in which only the antibody complementarity-determining regions (CDRs) are of non-human origin. Any of these antibodies, or fragments or derivatives thereof, are encompassed by the disclosure.

Antibody fragments and derivatives that recognize specific epitopes can be generated by any technique known to those of skill in the art. For example, Fab and F(ab′)₂ fragments of the disclosure can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies, or fragments or derivatives thereof, of the present disclosure can also be generated using various phage display methods known in the art. Finally, the antibodies, or fragments or derivatives thereof, can be produced recombinantly according to known techniques.

It will be apparent to the skilled practitioner that the affinity reagents can comprise binding domains other than antibody-based domains, such as peptidobodies, antigen-binding scaffolds (e.g., DARPins, HEAT repeat proteins, ARM repeat proteins, tetratricopeptide repeat proteins, and other scaffolds based on naturally occurring repeat proteins, etc. [see, e.g., Boersma and Pluckthun, Curr. Opin. Biotechnol. 22:849-857, 2011, and references cited therein, incorporated herein by reference]), which include a functional binding domain or antigen-binding fragment thereof.

As used herein, the term “treat” refers to medical management of a disease, disorder, or condition (e.g., autoimmune disease, rheumatic disease, IC-related inflammation, etc.) of a subject (e.g., a human or non-human mammal, such as another primate, horse, dog, mouse, rat, guinea pig, rabbit, and the like). Treatment can encompasses any indicia of success in the treatment or amelioration of a disease or condition (e.g., rheumatic disease or IC-related inflammation), including any parameter such as abatement, remission, diminishing of symptoms or making the disease or condition more tolerable to the subject, slowing in the rate of degeneration or decline, or making the degeneration less debilitating. Specifically in the context of inflammation, the term treat can encompass reducing inflammation, reducing pain associated with inflammation, or reducing the likelihood of recurrence, compared to not having the treatment. The treatment or amelioration of symptoms can be based on objective or subjective parameters, including the results of an examination by a physician. Accordingly, the term “treating” includes the administration of the compositions of the present disclosure to alleviate, or to arrest or inhibit development of the symptoms or conditions associated with disease or condition (e.g., autoimmune disease, rheumatic disease, IC-related inflammation, etc.). The term “therapeutic effect” refers to the amelioration, reduction, or elimination of the disease or condition, symptoms of the disease or condition, or side effects of the disease or condition in the subject. The term “therapeutically effective” refers to an amount of the composition that results in a therapeutic effect and can be readily determined.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

One of skill will recognize that individual substitutions, deletions or additions to a peptide, polypeptide, or protein sequence which alters, adds or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:

(1) Alanine (A), Serine (S), Threonine (T),

(2) Aspartic acid (D), Glutamic acid (E),

(3) Asparagine (N), Glutamine (Q),

(4) Arginine (R), Lysine (K),

(5) Isoleucine (I), Leucine (L), Methionine (M), Valine (V), and

(6) Phenylalanine (F), Tyrosine (Y), Tryptophan (W).

Reference to sequence identity addresses the degree of similarity of two polymeric sequences, such as protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are provided for the purpose of illustrating, not limiting, the disclosure.

### Example 1

This example describes a study of signaling mechanisms that direct neutrophil activity. The study was published in Lood, C., et al., 2017. “TLR7/8 activation in neutrophils impairs immune complex phagocytosis through shedding of FcgRIIA”, Journal of Experimental Medicine, 214(7):2103-2119, incorporated herein by reference in its entirety.

The study reports the finding that neutrophil TLR7/8 activation shifts neutrophils from phagocytosis of immune complexes to NETosis. Reduced phagocytosis of immune complexes is associated with partial proteolytic cleavage of FcgRIIA. Cleaved FcgRIIA is found in SLE neutrophils ex vivo.

### Abstract

Neutrophils play a crucial role in host defense. However, neutrophil activation is also linked to autoimmune diseases such as systemic lupus erythematosus (SLE) where nucleic acid-containing immune complexes (IC) drive inflammation. The role of Toll-like receptor (TLR) signaling in processing of SLE ICs and downstream inflammatory neutrophil effector functions is not known. We observed that TLR7/8 activation leads to a furin-dependent proteolytic cleavage of the N-terminal part of FcgRIIA shifting neutrophils away from phagocytosis of ICs toward the programmed form of necrosis, NETosis. TLR7/8 activated neutrophils promoted cleavage of FcgRIIA on plasmacytoid dendritic cells and monocytes resulting in impaired overall clearance of ICs and increased complement C5a generation. Importantly, ex vivo derived activated neutrophils from SLE patients demonstrated a similar cleavage of FcgRIIA that was correlated with markers of disease activity as well as complement activation. Therapeutic approaches aimed at blocking TLR7/8 activation would be predicted to increase phagocytosis of circulating ICs while disarming their inflammatory potential.

### Introduction

Neutrophils are the most abundant immune cells in the circulation, participating in host defense through mechanisms including production of reactive oxygen species (ROS), phagocytosis and formation of neutrophil extracellular traps (NETs), a neutrophil cell death process in which DNA is extruded together with cytoplasmic and granular content to eliminate extracellular pathogens. Although beneficial from a host-pathogen perspective, exaggerated neutrophil activation has been linked to autoimmunity, in particular the rheumatic disease systemic lupus erythematosus (SLE). In SLE, neutrophil abnormalities were described more than 50 years ago with the discovery of the lupus erythematosus cell (LE cell), a neutrophil engulfing IgG- and complement-opsonized nuclear debris. Circulating nucleic acid-containing immune complexes (ICs) participate in the SLE pathogenesis through activation of FcgR, complement and also by engaging intracellular TLR. Recently, we demonstrated that RNP containing ICs cause neutrophils to release interferogenic oxidized mitochondrial DNA during NETosis.

TLR agonists, such as nucleic acids, are important components of pathogens, enabling enhanced phagocytosis by macrophages and dendritic cells, as well as inducing cell maturation associated with a shift from phagocytosis to antigen presentation. Human neutrophils express all TLRs except for TLR3, with TLR8 rather than TLR7 being the most highly expressed single stranded RNA receptor. Nevertheless, the role of TLR signaling in neutrophil phagocytosis of SLE ICs and their downstream effects has not been extensively investigated. In this study, we reveal a novel mechanism in which TLR7/8 signaling, through shedding of FcgRIIA, shifts neutrophil function from phagocytosis to a programmed necrosis pathway, NETosis. The reverse was also true, namely, that phagocytic engagement decreased subsequent NET formation, suggesting neutrophil commitment to either NETosis or phagocytosis dependent on the environmental trigger. Finally, this process is clinically relevant as SLE patients had evidence for ongoing shedding of FcgRIIA related to neutrophil activation and markers of disease activity.

### Results

FcgR and TLR Cross-Talk Regulates Phagocytosis of RNP-ICs

IC-mediated neutrophil effector functions are thought to play a central role in the lupus pathogenesis. However, mechanisms regulating IC-mediated phagocytosis by neutrophils, and the specific contributions of FcgR- and TLR-engagement in this process, have not been studied in detail. Using ICs consisting of SmRNP and SLE IgG (RNP-ICs), shown previously to induce NETosis and specific FcgR-blocking monoclonal antibodies, we found that both FcgRIIA and FcgRIIIB were essential for RNP-IC-mediated phagocytosis, while FcgRI was dispensable, consistent with the low expression of FcgRI on resting neutrophils. Specifically, neutrophils were incubated with antibodies against FcgRs prior to stimulation with RNP-ICs. Phagocytosis was quantified by flow cytometry and compared to isotype antibody added (% of control). The experiment was repeated three times; combined results were compared using paired t test (P=0.013, P<0.0001, and P=0.0009 for FcgRI, FcgRIIA and FcgRIIIB, respectively). In contrast to studies done in transgenic cell lines and mice with rabbit IgG, we did not find any evidence of an FcgRIIA-independent role of FcgRIIIB in human neutrophils.

We next asked whether TLR7/8 activation, mediated through the RNA component of the RNP-ICs, influenced the phagocytosis of RNP-ICs by neutrophils. Specifically, TLR7/8 activation was inhibited by RNase or TLR7-9 iODN treatment prior to incubation of RNP-ICs with neutrophils, and phagocytosis analyzed by flow cytometry. The experiment was repeated three times (ODN) or six times (RNase); combined results are compared using paired t test (P=0.015, P=0.0006, and P=0.014 for SLE IgG, huRNase, and TLR7-9 iODN, respectively). Contrary to expectations, degradation of the TLR ligand by RNase resulted in an increase in the phagocytosis of RNP-ICs by neutrophils. This could not simply be explained by occupancy of the RNase-Fc dimer to FcgRIIA, which is prevented by the P283 S mutation, or by changes in the size or character of the RNP-IC because a similar observation was made when TLR activation was inhibited with a TLR7-9 inhibitory oligodeoxynucleotide (iODN). To establish this, neutrophils were incubated with human (hu)RNase or HAGG and analyzed for IgG-Fc binding by flow cytometry. The experiment was repeated three times. To determine whether the reciprocal was true, namely, that TLR activation could inhibit phagocytosis of ICs, the uptake of RNase-treated RNP-ICs was analyzed in presence of the TLR7/8 agonist, R848. Addition of R848 significantly decreased uptake of ICs as well as heat-aggregated IgG (HAGG). Specifically, neutrophils were stimulated with R848 prior to incubation with RNase-treated RNP-ICs, HAGG, beads or zymosan. The results are expressed as phagocytosis as compared to no R848 added (% of control). The experiment was repeated six (zymosan), eight (RNP-IC+RNase), nine (HAGG), or ten (beads) times; combined results were compared using paired t test (P=0.0005, P=0.0001, P<0.0001, and P=0.017 for RNP-IC+RNase, HAGG, beads, and zymosan respectively). This supports the hypothesis that TLR activation reduces FcgR-mediated phagocytosis in neutrophils. However, this process was selective—in contrast to ICs, TLR7/8 activation increased uptake of beads and zymosan. Finally, to determine if TLR7/8 activation affected the internalization process and/or the binding ability of the ICs, neutrophils were treated with the cytoskeleton inhibitor Cytochalasin B prior to adding the ICs, thus blocking uptake, but not binding. Specifically, neutrophils, treated with or without R848 followed by cytochalasin B (CytoB, 5 μM), were analyzed for binding and uptake of RNP-ICs by flow cytometry. The experiment was repeated six times; combined results were compared using paired t test (P<0.0001 for IC vs IC+CytoB, P=0.0066 for IC vs IC+R848, P=0.0078 for IC+CytoB vs IC+R848+CytoB, and P=0.0158 for IC+R848 vs IC+R848+CytoB). It was demonstrated that TLR7/8 activation suppressed both IC-mediated binding and subsequent phagocytosis indicating reduced FcgRIIA function.

TLR7/8 Activation Induces Selective Shedding of FcgRIIA

To determine the mechanism for the TLR-induced reduction in RNP-IC phagocytosis, we analyzed the neutrophil surface expression of FcgRs after exposure to TLR ligand. Neutrophils were activated with R848 and cell surface expression of FcgRs analyzed by flow cytometry. The results were presented as FcgR levels as compared to no R848 added (% of control). The experiment was repeated five (FcgRI), seven (FcgRIII), and twenty-five (FcgRIIA) times; combined results were compared using paired t test (FcgRIIA, P<0.0001; FcgRI, P=0.027; FcgRIII, P=0.0044). The expression of FcgRIIA was significantly reduced, whereas surface levels of FcgRIIIB and FcgRI were increased following TLR7/8 stimulation. The decrease in FcgRIIA surface expression was time- and dose-dependent. In this experiment, neutrophils were activated with the TLR7/8 agonist R848 and analyzed for FcgRIIA at different time-points and concentrations. The experiment was repeated four (concentration) and six (kinetics) times; combined results were compared using paired t test (30 min, P=0.0158; 60 min, P<0.0001; 120 min, P=0.0003; 0.125 μg/mL, P=0.0071; 0.25 μg/mL, P=0.0058; 0.5 μg/mL, P=0.0008; 1 μg/mL, P<0.0001; 2 μg/mL, P<0.0001). Loss of FcgRIIA was not specific for TLR7/8 engagement as neutrophil incubation with either TLR1/2, TLR4, TLR7, or TLR8 selective agonists also reduced neutrophil cell surface levels of FcgRIIA, but not of FcgRIIIB, concomitant with increased expression of CD11b and CD66b. For these experiments, neutrophils were activated with TLR ligands (LPS, 1 μg/mL, PAM3CSK4 (5 μg/mL), CpG DNA (2 μg/mL), Loxoribine (0.1 mM), CL075 (2.5 μg/mL) or R848 (2 μg/mL)) for 60 minutes or 4 hours and analyzed for FcgRIIA, FcgRIII or CD11b and CD66b cell surface expression by flow cytometry. Experiments were repeated six (LPS, P=0.0008), eight (CpG DNA, P=0.035; Loxoribine, P<0.0001, and CL075, P<0.0001), ten (PAM3CSK4, P<0.0001) and forty times (R848, P<0.0001); combined results were compared using paired t test. Additional experiments were repeated four and eight times; combined results were compared using paired t test (CD11b: R848, P<0.0001; LPS, P=0.0002; PAM, P<0.0001; CpG DNA, P<0.0001; CD66b: R848, P<0.0001; LPS, P<0.0001; PAM, P<0.0001; CpG DNA, P=0.014). Similar results were also seen with PMA.

To assess if reduction in FcgRIIA cell surface expression was dependent on proteolytic cleavage or internalization of the receptor, we analyzed total FcgRIIA expression in fixed permeabilized neutrophils. Specifically, neutrophils were activated with R848 and FcgRIIA levels analyzed in permeabilized cells by flow cytometry. The experiment was repeated five times and compared using paired t test (P=0.0075). Similar to cell surface staining, R848 reduced the overall FcgRIIA levels in neutrophils. Reduced expression was only seen with one of the antibody clones tested (IV.3, recognizing amino acid 132-137), but not with the FUN2 clone, indicating that only the most N-terminal part of the FcgRIIA was lost, rather than the full receptor. Specifically, FcgRIIA cell surface expression was analyzed by flow cytometry using two antibodies, FUN2 and IV.3, in non-stimulated and R848-stimulated neutrophils. The experiment was repeated six times; combined results were compared using paired t test (P<0.0001). Furthermore, using cells to which anti-FcgRIIA antibodies had been added (‘pre-labeled’), FcgRIIA-IV.3 complexes, but not FcgRIIA-FUN2 complexes, were detected in increased amounts in the cell-free supernatant upon R848 activation compared to non-stimulated cells. For this experiment, neutrophils were labeled with FITC-conjugated IV.3 anti-FcgRIIA or anti-FUN-2 antibodies and the shed antibody-FcgRIIA complex quantified by fluorimetry following R848 stimulation with or without prior addition of a pan-protease inhibitor. The experiment was repeated four (FUN2), six (IV.3 R848+prot.inh.) or fourteen (IV.3 R848) times; combined results were compared using paired t test (IV.3: R848, p<0.0001; R848+prot.inh. P=0.0001). Addition of a pan protease inhibitor markedly reduced the overall accumulation of cell-free FcgRIIA-anti-CD32A complexes in the supernatant, indicating that proteolytic cleavage of cell surface FcgRIIA was responsible for reduced FcgRIIA expression following TLR7/8 engagement. The ability of the protease inhibitor to reduce the amount of shed FcgRIIA even further than baseline suggests basal shedding activity of the neutrophil also occurs in the resting state.

To determine which protease(s) were involved in the shedding of FcgRIIA, neutrophils were incubated with selective protease inhibitors prior to the addition of the TLR agonist. Cell surface levels of FcgRIIA (IV.3) was analyzed by flow cytometry upon R848 activation in the presence of a pan protease inhibitor or inhibitors of matrix metalloproteases (GM6001, 10 μM), cysteine proteases (E-64, 1 μM), serine proteases (AEBSF, 100 μM), neutrophil elastase (Elastase inhibitor IV, 25 μM), cathepsin G (chymostatin, 10 μg/mL) or furin (chloromethylketone (CMK, 25 μM). The experiment was repeated three (E-64), four (Pan Prot.inh., P<0.0001; AEBSF, P=0.0004; chymostatin; and CMK, P=0.0038), five (GM6001), and seven (NEi) times; combined results were compared using paired t test. TLR7/8-mediated shedding of FcgRIIA was dependent on serine proteases, including the pro-protein convertase furin. Additionally, neutrophils were incubated with furin (100 ng/mL) or CMK 30 minutes prior to addition of R848. BAFF cell surface expression was analyzed by flow cytometry. The experiment was repeated seven times; combined results were compared using paired t test (R848, P=0.0095; R848+Furin, P=0.0027; R848+CMK, P=0.002). Although addition of recombinant furin increased cell surface BAFF levels, exogenously added furin did not affect FcgRIIA shedding on neutrophils. Briefly, neutrophils were incubated with furin (100 ng/mL) in presence or absence of R848 and analyzed for FcgRIIA levels by flow cytometry. The experiment was repeated nine times. Finally, supernatant from activated neutrophils was fractionated and analyzed for capacity to induce shedding of monocyte FcgRIIA without, or with prior boiling of the fractions. For the experiment without prior boiling of the fractions, the 30 kDa pool was used. The experiment was repeated four (30 kDa pool), six (10 kDa and 100 kDa), or seven (30 kDa fractions) times; combined results were compared using paired t test (>30 kDa, P=0.0003; <30 kDa, P=0.0015; 30 kDa pool, P=0.016 and P=0.018 as compared to supernatant and <30 kDa fraction respectively; >10 kDa, P<0.0001; <10 kDa, P=0.0002; >100 kDa, P=0.0001). For the experiment with prior boiling of the fractions, the experiment was repeated three (30 kDa fraction and pool) or six (boiled supernatant) times; combined results were compared using paired t test (boiled supernatant, P=0.0035; Boiled >30 kDa, P=0.017; Boiled <30 kDa, P=0.011). Thus, furin most likely did not act directly on FcgRIIA but on an intracellular process. Although the protease(s) that cleaves FcgRIIA remains to be identified, we found the neutrophil supernatant to require both a small (<10 kDa) heat-sensitive component, as well as a larger (30-100 kDa) protein to induce shedding of FcgRIIA.

FcgRIIA Shedding Requires PI3K-Dependent Generation of Reactive Oxygen Species

Neutrophils were activated with R848, and FcgRIIA and CD66b levels analyzed by flow cytometry. The experiment was repeated eight times; combined results were compared using paired t test (P<0.0001). It was shown that FcgRIIA shedding was associated with the most activated neutrophils. Thus, we applied a phosphoproteomic mass spectrometry-based approach to identify proteins and pathways activated by R848 and RNP-ICs that could contribute to shedding of FcgRIIA. A heat-map was generated illustrating phosphoproteins modified upon TLR7/8 activation by R848 and RNP-ICs. Results were expressed as fold change as compared to non-stimulated neutrophils with green representing decreased phosphorylation and red indicating increased phosphorylation. Amongst the identified phosphoproteins, several were involved in cytoskeletal regulation (ADD1, LSP1, VIM and SYNE1), exocytosis (STXBPS), or MAPK signaling (MAPK14), consistent with the KEGG analysis (Table 1).

TABLE 1
KEGG pathway analysis upon TLR7/8 activation

| KEGG pathway | P-value |
|---|---|
| FcgR-mediated phagocytosis | p = 0.00014 |
| Regulation of actin cytoskeleton | p = 0.0035 |
| Endocytosis | p = 0.032 |
| MAPK signaling pathway | p = 0.043 |

Another target of TLR7/8 stimulation was ncf1 (p47 phox). Ncf1 was phosphorylated at S345, a known target site involved in activation of the NADPH oxidase complex. Briefly, ncf1 was phosphorylated (p47 phox) at S345 upon R848 activation as determined by phosphoproteomics. The experiment was repeated three times; combined results were compared using paired t test (P=0.044). Neutrophils were incubated with R848 in the absence or presence of the PI3K inhibitor Ly294002 and analyzed for pS345 or total levels of p47 phox using Western Blot. The experiment was repeated four times; combined results were compared using paired t test (No stim, P=0.03; R848+LY294002, P=0.0011). As ROS increases the sensitivity of target proteins for proteolytic degradation as well as activates redox-sensitive proteases, we asked if ROS generation was necessary for shedding of FcgRIIA. Addition of either DPI or apocynin, two well-established inhibitors of NADPH oxidase, completely restored cell surface levels of FcgRIIA. Briefly, neutrophils were treated with inhibitors of NADPH oxidase prior to addition of R848 and analyzed for cell surface expression of FcgRIIA by flow cytometry. The experiment was repeated six times; combined results were compared using paired t test (DPI, P=0.0042; Apocynin, P=0.0044). Inhibiting ROS also increased the cell surface expression of FcgRIIIB upon TLR7/8 activation, albeit only modestly, suggesting that both FcgRs are negatively regulated through a ROS-dependent mechanism. Consistent with those results, neutrophils from CGD patients, deficient in NADPH oxidase-mediated ROS production, did not show reduced cell surface levels of FcgRIIA upon TLR7/8 engagement, despite CGD neutrophils being able to up-regulate cell surface activation marker, CD66b. Briefly, neutrophils from healthy individuals (HV, n=18) and CGD patients (n=4) were stimulated with R848 and analyzed for FcgRIIA levels by flow cytometry. The data were analyzed using paired t test (HC, P<0.0001) and unpaired t test (HC vs CGD, P=0.0097). Neutrophils from healthy individuals (HV, n=13) and CGD patients (n=3) were activated by R848 and analyzed for CD66b expression by flow cytometry using paired t test (HC, P<0.0001; CGD, P=0.039). TLR1/2 and TLR4-mediated shedding of FcgRIIA was also dependent on NADPH oxidase, suggesting a similar signaling pathway being involved for all TLR agonists. Briefly, neutrophils were activated with LPS or PAM3CSK4 in presence of DPI and analyzed for FcgRIIA levels by flow cytometry. The experiment was repeated four times; combined results were compared using paired t test (LPS, P=0.0062; PAM, P=0.0003). To determine if TLR7/8-mediated ROS was generated intracellularly, or released extracellularly by plasma membrane-located NADPH oxidase complexes, we analyzed the cellular localization of ROS using cell impermeable ROS dyes as well as flow cytometry. Briefly, neutrophils were activated with R848, RNP-ICs, or PMA and analyzed for cellular localization for the ROS generation by flow cytometry and fluorimetry. The experiment was repeated five (extracellular) and eight (intracellular) times; combined results were compared using paired t test (Extracellular: PMA, P=0.007; Intracellular: PMA, P<0.0001; RNP-IC, P=0.0007; R848, P<0.0001). Both R848 and RNP-ICs induced intracellular generation of ROS, but no detectable extracellular ROS, whereas PMA induced both intracellular and extracellular ROS generation, suggesting formation of endosomal, but not cell surface, NADPH oxidase complexes following stimulation with RNP-ICs and R848.

We next asked which pathway(s) were acting upstream of NADPH oxidase to induce FcgRIIA shedding. Several regulators of NADPH oxidase have been demonstrated, amongst which PI3K is central, and known to be essential in IC-mediated neutrophil activation. Phosphorylation of Akt and S6 was determined by flow cytometry upon TLR7/8 activation. The experiment was repeated four times; combined results were compared using paired t test (pS6, P=0.042; pAkt, P=0.037). Neutrophil TLR7/8 ligation induced increased levels of phosphorylated Akt and S6 as determined by flow cytometry, and S6 was one of the most phosphorylated proteins as determined by phosphoproteomics, strongly suggesting PI3K activation upon TLR7/8 activation. To confirm the role for PI3K in TLR-mediated activation of ROS and subsequent shedding of FcgRIIA, neutrophils were incubated with the PI3K inhibitor LY294002 prior to addition of TLR agonist. Blocking PI3K signaling abrogated TLR-mediated ROS generation, phosphorylation of ncf1 at S345 as well as shedding of FcgRIIA. Briefly, neutrophils, pre-treated with inhibitors of PI3K (LY294002, 10 μM) or NADPH oxidase (DPI, 25 μM), were activated with R848 and analyzed for ROS generation by flow cytometry using DHR123. The experiment was repeated three times; combined results were compared using paired t test (R848, P=0.0049; R848+LY294002, P=0.004; R848+DPI, P=0.0031). Additionally, neutrophils were pre-treated with the PI3K inhibitor LY294002 (10 μM) and analyzed for R848-mediated shedding of FcgRIIA by flow cytometry. The experiment was repeated eight times; combined results were compared using paired t test (P<0.0001).

Also, heat-aggregated IgG (HAGG) cross-linking of FcgRIIA activated neutrophils to induce shedding of FcgRIIA in a PI3K-dependent manner, albeit to a smaller extent than TLR activation. For these assays, neutrophils, with or without pre-treatment with LY294002, were activated with heat-aggregated IgG (HAGG) and analyzed for CD66b, FcgRIIA shedding, and pS6 expression by flow cytometry. Combined results were compared using paired t test (R848, P<0.0001; HAGG, P=0.017; R848 vs HAGG, P=0.0001), (R848, P<0.0001; HAGG, P=0.0002; R848 vs HAGG, P=0.0029; HAGG vs HAGG+LY294002, P=0.018), and (HAGG, P=0.0024; R848, P=0.0314; RNP-IC, P=0.011), respectively. Taken together, these data demonstrate that PI3K-driven ROS production via NADPH oxidase is necessary for TLR7/8-mediated shedding of FcgRIIA.

TLR7/8-Mediated Shedding of FcgRIIA Shifts Neutrophil Function from Phagocytosis to NETosis

Given the ability of TLR7/8 to induce shedding of FcgRIIA, we asked what the biological consequences of FcgRIIA shedding on neutrophil key effector functions would be. Neutrophils were incubated with CMK (25 μM), prior to addition of stimuli and phagocytosis analyzed by flow cytometry. The experiment was repeated five times; combined results were compared using paired t test (P=0.0032). As expected, adding the furin inhibitor we observed a selective increase in the uptake of RNP-ICs, but not of latex beads, consistent with a role for furin in promoting FcgRIIA shedding. Additionally, neutrophils were incubated with CMK prior to the addition of RNP-ICs and cell surface levels of CD11b and CD66b analyzed by flow cytometry. The results were expressed as CD11b or CD66b (% of control) as compared to non-stimulated cells. The experiment was repeated fifteen times; combined results were compared using paired t test (CD11b: RNP-IC; P<0.0001; RNP-IC+CMK, P=0.0002; CD66b: RNP-IC; P<0.0001; RNP-IC+CMK, P<0.0001). It was demonstrated that the furin inhibitor also amplified RNP-IC-mediated neutrophil activation. However, in contrast to increased phagocytosis, addition of CMK decreased RNP-IC-mediated NETosis (Neutrophils, pre-treated with CMK, were activated with RNP-ICs and the ability to release NETs analyzed by fluorimetry. The experiment was repeated six times; combined results are compared using paired t test (P=0.0001)). A similar phenomenon was observed using RNase treatment of RNP-ICs. Removal of the RNA component increased phagocytosis, but reduced NETosis (RNP-ICs were treated with RNases prior to addition to neutrophils and NET formation analyzed by fluorimetry. The experiment was repeated seven times; combined results were compared using paired t test (P<0.0001)), indicating opposite regulation of RNP-IC-mediated phagocytosis and NETosis in neutrophils. Importantly, RNase did not degrade the NETs. RNase-mediated degradation of RNA in the SmRNP complex was also observed in the presence of anti-Sm/RNP autoantibodies. Briefly, SmRNP, NETs, dsDNA or ssRNA were degraded by human RNase without or with presence of autoantibodies, and analyzed by fluorimetry over time.

Since we observed contrasting effects with regard to TLR7/8 stimulation limiting phagocytosis while promoting NETosis, we asked if phagocytosis and NETosis were opposing processes in neutrophils. In support of this hypothesis, we found that addition of beads that stimulated phagocytosis inhibited RNP-IC-mediated NETosis in a dose-dependent manner (NET formation was analyzed upon pre-incubation with different amounts of beads; the experiment was repeated three times; combined results were compared using paired t test (1 μL, P=0.0135; 5 μL, P=0.0031)). Addition of beads did not hinder subsequent uptake of RNP-ICs. On the contrary, neutrophils primed with phagocytic stimuli (beads) had an enhanced ability to phagocytose RNP-ICs, while losing the capacity to undergo NETosis (neutrophil uptake of RNP-ICs was analyzed upon pre-treatment with beads; the experiment was repeated four times; combined results were compared using paired t test (P=0.018)). Importantly, in neutrophils from healthy controls, high levels of full-length FcgRIIA were associated with an increased phagocytic ability, but decreased NET forming capacity of the neutrophils (neutrophils from healthy individuals (n=12) were analyzed for baseline FcgRIIA IV.3/FUN2 ratio in relation to IC-mediated NETosis and phagocytosis; the combined results were analyzed using Spearman's correlation), further verifying the inverse regulation between IC-mediated phagocytosis and NETosis. Thus, we have identified a novel process in which neutrophil function, through TLR7/8-mediated shedding of FcgRIIA, shifts from phagocytosis to NETosis. Importantly, the reverse also seems to be true, e.g., when neutrophils commit to phagocytosis they reduce their NET-inducing capacity.

Activation of Neutrophil TLR7/8 Results in Proteolytic Cleavage of FcgRIIA on Monocytes and pDCs as Well as a Reduction in Monocyte Phagocytosis

Since we observed prominent protease-mediated shedding of FcgRIIA in neutrophils, we next asked if activated neutrophils could induce shedding of FcgRIIA in other immune cells. PBMCs were co-incubated with neutrophils (PMNs) in the presence of R848 and a pan-protease inhibitor. Levels of FcgRIIA on monocytes (CD14+), and pDCs (CD304+) were determined by flow cytometry and expressed as FcgRIIA (% of control) as compared to PBMCs incubated in medium in absence of neutrophils. The monocyte experiment was repeated eleven times with the exception of the proteinase inhibitor (n=5); combined results were compared using paired t test (PMN+PBMC, P<0.0001; PMN+PBMC vs PMN+PBMC+R848, P<0.0001; PMN+PBMC+R848 vs PMN+PBMC+R848+Prot.inh., P=0.002). The pDCs experiment was repeated seven times; combined results were compared using paired t test (PMN+PBMC, P=0.0261; PMN+PBMC vs PMN+PBMC+R848, P=0.0002; PMN+PBMC+R848 vs PMN+PBMC+R848+Prot.inh., P=0.0128). Although R848 induced monocyte activation and up-regulation of cell surface CD11 b, monocyte surface expression of FcgRIIA was unchanged. However, upon co-culture with neutrophils primed with R848, monocytes lost cell surface FcgRIIA expression in a protease-dependent manner. Similar findings were observed in pDCs, with loss of FcgRIIA in a neutrophil- and protease-dependent manner. Comparable to what was observed in neutrophils, the loss of FcgRIIA expression on monocytes was selective for the IV.3 clone, since neither the expression of the FUN2 epitope nor FcgRI was altered indicating a similar protease was operative (monocytes were analyzed for the expression of FcgRI (CD64) as well as FcgRIIA using the monoclonal antibodies IV.3 and FUN2; the experiment was repeated four times; combined results were compared using paired t test (P=0.008)). However, FUN-2 also targets FcgRIIB, although expressed at much lower levels than FcgRIIA on monocytes.

To determine if the loss of monocyte FcgRIIA was mediated through cell-cell interactions or due to a soluble neutrophil-derived factor, we added supernatant from TLR7/8-activated neutrophils to monocytes. Specifically, neutrophil supernatant, derived from non-stimulated (no, n=5) or R848-stimulated (R848, n=9) neutrophils, were added to monocytes in presence of indicated inhibitors (CMK; furin inhibitor, n=5, and Pan; pan-protease inhibitor, n=5) and monocyte FcgRIIA levels analyzed by flow cytometry. Combined results were compared using paired t test (R848, P<0.0001; R848+Pan, P=0.003). Cell-free supernatant from R848-activated neutrophils reduced monocyte FcgRIIA levels, indicating the presence of a soluble neutrophil factor able to mediate shedding of monocyte FcgRIIA. The neutrophil supernatant shed monocyte FcgRIIA in a protease-dependent, but furin-independent manner—further demonstrating that furin does not act directly on FcgRIIA. Importantly, similar to what was observed in neutrophils stimulated directly or in the neutrophil-PBMC co-culture experiments, the supernatant derived from TLR7/8 activated neutrophils resulted in the selective shedding of the N-terminal region of the FcgRIIA (neutrophil supernatant was added to monocytes and expression of FcgRIIA (IV.3 and FUN2) as well as FcgRI (CD64) analyzed by flow cytometry; the experiment was repeated four times; combined results were compared using paired t test (P=0.0043)). Attempting to characterize the shed FcgRIIA by Western blot, recombinant FcgRIIA was incubated with neutrophil supernatant to cleave the receptor. Similar to what was found for the immune cells, addition of neutrophil supernatant led to a clear reduction in overall levels of full-length FcgRIIA. However, no low molecular fragment was observed either upon probing with clone IV.3 or using biotinylated FcgRIIA, suggesting that the degraded peptides were too small to be detected by Western blot. Although unlikely, considering the inability of R848 to induce shedding of FcgRIIA on monocytes and pDCs in PBMC cultures, an indirect role of another PBMC subset in mediating neutrophil-dependent shedding of monocyte and pDC FcgRIIA could not be ruled out.

As neutrophil proteases released after TLR activation promoted loss of FcgRIIA from monocytes and pDCs, we next examined the functional consequences of shedding. Monocytes were incubated with R848 or neutrophil supernatant prior to addition of RNP-ICs or beads. Phagocytosis was determined by flow cytometry. The experiment was repeated four (beads, RNase, RNP-IC+R848) or seven (PMN sup) times; combined results were compared using paired t test (RNP-IC, P=0.0003; Beads, P=0.0007). Whereas monocyte phagocytosis of RNP-ICs was not affected by exposure to RNase or by priming with R848, addition of neutrophil supernatant decreased monocyte phagocytosis of RNP-ICs by more than 50%. To determine whether the reduction in IC phagocytosis impacted complement activation, we quantified release of the complement split product, C5a, by ELISA and observed that the reduced clearance of ICs induced increased generation of C5a (ICs were added to PBMCs with or without prior treatment with neutrophil supernatant (see above); after phagocytosis for 30 minutes, remaining cell-free ICs were analyzed for C5a-inducing ability upon addition of 1% normal human serum; the experiment was repeated three times; combined results were compared using paired t test (P=0.0084)). This anaphylatoxin is known to promote inflammation and recruitment of immune cells, in particular neutrophils. Consistent with this finding, SLE patients had increased C5a levels which correlated with shedding of neutrophil FcgRIIA. Briefly, C5a serum levels were measured in healthy controls (HC, n=9) and SLE patients (n=36) by ELISA. Combined results were analyzed using Mann-Whitney U test (P=0.047). Serum levels of C5a in SLE patients were related to ability of serum to induce shedding of FcgRIIA on healthy control neutrophils. Combined results from 35 SLE patients were analyzed using Spearman's correlation test (r=−0.42, P=0.011), or combined results from SLE patients inducing shedding (n=15) or not (n=20), were compared using Mann-Whitney U test (P=0.0281). Thus, we propose that neutrophil mediated shedding of FcgRIIA on immune cells results in reduced FcgRIIA mediated IC clearance in vivo. In normocomplementemic individuals, early complement components (C1q, C3) may provide a non-inflammatory pathway for clearance. However, in SLE patients who frequently have low levels of classical complement pathway components, activation and generation of C5a may lead to deleterious consequences.

Selective FcgRIIA Shedding is Present in SLE Patients and Correlated with Neutrophil Activation

To investigate the potential clinical relevance of our observations, we analyzed cell surface levels of FcgRIIA on neutrophils and monocytes from SLE patients, a disease where neutrophil abnormalities have been reported previously by us and others. Using the same two antibody clones to detect either full-length receptor (IV.3) or total levels (FUN2), we observed that neutrophils and monocytes from SLE patients demonstrated reduced expression of the most N-terminal portion of FcgRIIA as compared to healthy individuals (neutrophils and monocytes were analyzed for FcgRIIA shedding using a ratio between shed FcgRIIA (IV.3) and total FcgRIIA levels (FUN2) in healthy controls (HC, n=5-7) and SLE patients (n=19); combined results were compared using Mann-Whitney U test (Neutrophils, P<0.0001; Monocytes, P<0.0001)). Interestingly, low-density granulocytes (LDGs), known to spontaneously release NETs, had a greater degree of FcgRIIA shedding compared to their normal-density counterparts (normal-density neutrophils (PMNs) and low-density granulocytes (LDGs) were analyzed for FcgRIIA shedding by flow cytometry; combined results from six SLE patients were compared using paired t test (P=0.026)). SLE-derived neutrophils were overall activated and importantly, patients having high neutrophil activation had the lowest IV.3/FUN2 ratio (neutrophil FcgRIIA shedding was correlated with neutrophil activation as measured by neutrophil CD11b and CD66b expression in SLE patients (n=19); combined results were analyzed using Spearman's correlation (CD66b: r=−0.64, P=0.0029; CD11 b: r=−0.53, P=0.021)), consistent with our in vitro studies. Thus, ex vivo, neutrophil activation is associated with loss of FcgRIIA on neutrophils and monocytes.

As neutrophil and monocyte FcgRIIA shedding was highly correlated in SLE patients upon ex vivo analysis (correlation analysis for ex vivo monocyte and neutrophil (PMN) FcgRIIA shedding in SLE patients was performed; combined results were analyzed using Spearman's correlation (r=0.84, P<0.0001)), we asked whether this could be attributed to circulating proteases, likely neutrophil-derived. The addition of SLE serum, but not serum from healthy controls, induced shedding of FcgRIIA on neutrophils (healthy control neutrophils were incubated with 10% serum from healthy controls (HC, n=10) or SLE patients (n=36) and analyzed for FcgRIIA shedding by flow cytometry as determined by the IV.3/FUN2 ratio; combined results were compared using Mann-Whitney U test (P<0.0001)), in a RNA- and protease-dependent manner (sera from 6 SLE patients, pre-incubated with either RNase, a pan-protease inhibitor (prot.inh.), or cytochalasin B (Cyto B, 5 μM) were added to neutrophils from a healthy individual and FcgRIIA shedding analyzed by flow cytometry; combined results were compared using paired t test (RNase: P=0.012; prot.inh.: P=0.0002; Cyto B: P<0.0001)), suggesting that the presence of both RNA ICs and proteases participated in the shedding of FcgRIIA as was shown using purified components. Consistent with a role of RNA ICs, serum-mediated FcgRIIA shedding was higher in patients with anti-Sm/RNP antibodies (sera from 6 SLE patients, pre-incubated with either RNase, a pan-protease inhibitor (prot.inh.), or cytochalasin B (Cyto B, 5 μM) were added to neutrophils from a healthy individual and FcgRIIA shedding analyzed by flow cytometry; combined results were compared using paired t test (RNase: P=0.012; prot.inh.: P=0.0002; Cyto B: P<0.0001)). To determine if serum-mediated shedding of FcgRIIA involved engulfment of RNP-ICs and subsequent de novo release of neutrophil proteases, healthy control neutrophils were incubated with a cytoskeletal inhibitor prior to addition of lupus sera. Addition of cytochalasin B almost completely abrogated serum-mediated FcgRIIA shedding, indicating that RNP-ICs needed to be internalized in order to promote shedding of FcgRIIA. Finally, SLE serum-mediated shedding of FcgRIIA from healthy control neutrophils strongly correlated with the FcgRIIA shedding observed upon ex vivo isolation of the SLE patient's neutrophils (correlation between ex vivo FcgRIIA shedding observed on SLE neutrophils with the shedding ability by the serum obtained from the same SLE patients (n=12); combined results were analyzed by Spearman's correlation (r=0.73, P=0.0096)). In summary, FcgRIIA on SLE monocytes and neutrophils demonstrate shedding at a site similar or identical to that identified by RNP-IC activated neutrophils in vitro, which can be attributed to RNA-ICs and proteases.

### Discussion

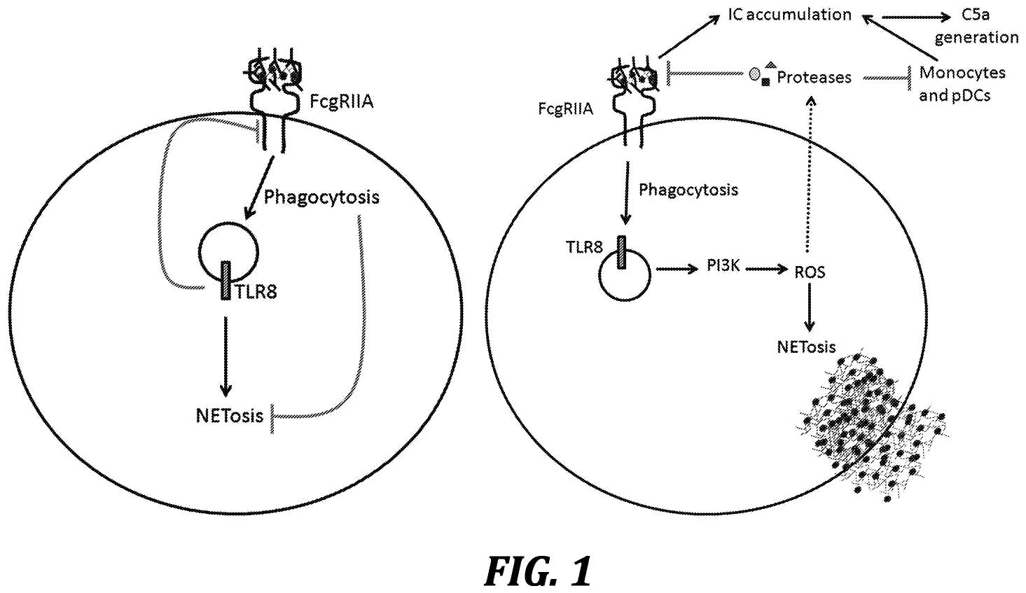

The precise mechanisms of how nucleoprotein-containing ICs impact recognition, phagocytosis and subsequent induction of neutrophil effector functions have not been well characterized. In the current investigation we made the novel finding that activation of TLR7/8, upon engulfment of RNP-ICs, induced proteolytic cleavage of FcgRIIA thereby shifting neutrophil function from phagocytosis of ICs to a program dedicated to NETosis. In contrast, when phagocytosis was increased by any one of three stimuli: blockade of TLR activation; inhibition of FcgRIIA shedding; or by priming neutrophils with a phagocytic stimulus, IC-mediated NETosis was markedly impaired. Together, these findings suggest an important cross regulation between phagocytosis and NETosis (FIG. 1). Our observations are consistent with the finding that phagocytosis of microbes led to a reduction in NETosis. Thus, we propose that, in a process analogous to what has been described for dendritic cells upon TLR activation, in which DCs lose their phagocytic capacity while gaining an effector function (antigen presentation), TLR7/8 stimulation by RNP-ICs leads to a reduction in subsequent IC phagocytosis and dedicates neutrophils to a terminal effector function, NETosis. Interestingly, patients with SLE, known to have exuberant NET formation, as well as decreased phagocytic ability, demonstrated substantial shedding of neutrophil FcgRIIA ex vivo, suggesting commitment of a proportion of their neutrophils towards the NET-inducing phenotype. Consistent with this interpretation, LDGs, that spontaneously generate NETs, had increased cleaved FcgRIIA as compared to their normal-density counterparts.

Loss of cell surface FcgRIIA has been described previously in human Langerhans cells as well as neutrophils upon fMLP-mediated activation, although the underlying mechanism(s) was not known. Following IC stimulation of neutrophils, we observed that only the most N-terminal portion of the FcgRIIA was shed as staining by the IV.3 antibody (that recognizes amino acids 132-137 of the second extracellular domain Ramsland, P. A., et al., 2011. Structural basis for Fc gammaRIIa recognition of human IgG and formation of inflammatory signaling complexes. J Immunol 187:3208-3217, incorporated herein by reference in its entirety) was lost, yet recognition by FUN2 (precise epitope not known) was retained. Enzyme inhibition studies implicated the pro-protein convertase, furin, as participating in the shedding of FcgRIIA, but this effect was not direct. Several other effects of furin may explain the action of this enzyme. There is a predicted furin cleavage site located at the junction of the transmembrane and intracytoplasmic domains so that intracellular furin cleavage could alter FcgRIIA conformation rendering it more susceptible to cleavage by another protease. Alternatively, or in addition, furin has been shown to be involved in the activation of several other proteases, including MMPs as well as ADAM10 and ADAM17. ADAM17 has been implicated in shedding of FcgRIIIB, but we were unable to inhibit FcgRIIA shedding by inhibitors of either MMPs or ADAM proteases. Furin may act even further upstream—furin-like proprotein convertases are essential in endosomal cleavage and subsequent activation of TLR7 and TLR8. Although we did not observe an effect of furin inhibition on proteolytic activation of TLR8 in neutrophils, we observed that inhibition of furin reduced TLR7/8-mediated ROS generation, which we showed here was necessary for FcgRIIa shedding. Further studies are needed to determine the furin substrates and which proteases other than furin are involved in the shedding of FcgRIIA.

Even though TLR7/8-mediated shedding of FcgRIIA was selective for neutrophils, transfer of neutrophil culture supernatant, or co-culture, enabled shedding of FcgRIIA on monocytes and pDCs, reducing their overall phagocytic ability. This resulted in increased generation of C5a, which promotes recruitment of neutrophils and macrophages, activation of phagocytic cells, release of granular proteins and generation of oxidants, all contributing to shaping the innate immunity and mediating tissue damage. Thus, we postulate that initial RNP-IC engagement of neutrophils promotes neutrophil maturation to NETosis as well as FcgRIIA shedding. By inducing shedding of FcgRIIA on adjacent immune cells, FcgRIIA-facilitated clearance of ICs as well as cytokine production are reduced whereas C5a facilitates recruitment of fresh phagocytes to remove ICs. In a normocomplementemic state, IC bound C3b will facilitate resolution through clearance mechanisms that are less inflammatory. However, in a hypocomplementemic state and/or with an abnormal CR3 (ITGAM) variants that impair clearance of IC by complement as occurs in SLE, persistent activation of the terminal complement pathways will contribute to persistent inflammation.

Since shedding of FcgRIIA was not selective for TLR7/8, but observed for most of the TLR agonists tested, we asked what common signaling pathways could be involved in regulating FcgRIIA shedding. We found that shedding of FcgRIIA was mediated through the PI3K pathway and subsequent activation of NADPH oxidase as demonstrated by the use of selective inhibitors as well as neutrophils obtained from CGD donors deficient in NADPH oxidase. Consistent with an impaired ability to undergo shedding of FcgRIIA in CGD patients, prior investigations have demonstrated an increased ability of CGD neutrophils to ingest ICs, although having similar baseline levels of FcgRIIA as healthy control neutrophils. This is of particular interest as patients with impaired ROS production, thus unable to shed FcgRIIA and subsequently will promote phagocytosis by monocytes and pDCs, develop a type I IFN signature with a risk of autoimmunity as observed in both SLE and CGD patients. Although the role of ROS in this process is yet not fully understood, ROS has been shown to increase the sensitivity of target proteins for proteolytic degradation as well as activate redox-sensitive proteases. However, it should be acknowledged that ROS may act through several pathways to regulate inflammation and autoimmunity, including induction of hypoxia, which modulates the host response to inflammation promoting resolution.

In conclusion, we have identified an intricate cross-talk between FcgRIIA and TLR7/8 that impacts phagocytosis and NETosis and unraveled several signal transduction pathways responsible. These observations extend our understanding of neutrophil function in regulation of autoimmunity and inflammation, and demonstrate that therapeutic interventions to prevent TLR7/8 activation would increase phagocytic clearance of ICs while limiting their ability to induce inflammatory NETosis.

### Material and Methods

Patients and Controls

All individuals signed informed consents in IRB-approved protocols (University of Washington; HSD number 39712). Pediatric samples from CGD individuals were obtained through the Seattle Children's Research Institute Center for Immunity and Immunotherapies Repository for Immune-Mediated Diseases.

NET Induction and Quantification

Human neutrophils were isolated by Polymorphprep™ (Axis-Shield) as described previously (Lood, C., et al., 2016. Neutrophil extracellular traps enriched in oxidized mitochondrial DNA are interferogenic and contribute to lupus-like disease. Nat Med 22:146-153; incorporated herein by reference in its entirety). Neutrophils (1×10⁶ cells/mL) were incubated in poly-L-lysine coated tissue culture plates with or without furin inhibitor chloromethylketone (CMK, 25 μM, Enzo Life Sciences), PI3K inhibitor LY294002 (10 μM, Invivogen), pan-caspase inhibitor Q-VD-Oph (10 μM, Sigma), R848 (1 μg/mL, Invivogen) or latex beads for 1 hour prior to addition of PMA (20 nM) or RNP-ICs (IgG, purified from SLE patients with high titer anti-ribonucleoprotein (RNP) antibodies, or healthy individuals, mixed with SmRNP (Arotec Diagnostic Limited) used at final concentration of 10 μg/mL). In some experiments, RNP-ICs were pre-treated with 0.25 mM human dimeric RNase-Fc for 30 minutes at 37° C. before being used. NETs were detached with micrococcal nuclease (0.3 U/mL, Fisher Scientific) diluted in nuclease buffer containing 10 mM Tris-HCl pH 7.5, 10 mM MgCl₂, 2 mM CaCl₂ and 50 mM NaCl. Detached NETs were quantified by analyzing Sytox Green (Life Technologies) intensity by plate reader (Synergy 2, BioTek).

Phagocytosis Assay

SLE IgG, SmRNP and heat-aggregated IgG (HAGG) were labeled with Alexa-647 according to manufacturer's protocol (Life Technologies). Neutrophils, or PBMCs, from healthy individual were stimulated with different ICs, FITC-conjugated latex beads or zymosan (100 μg/mL, Life Technologies) for 30 minutes at 37° C. and immediately analyzed for phagocytosis. In blocking experiments, neutrophils were incubated with 0.1 μM TLR7-9 or control iODN (Enzo Life Sciences), CMK (25 μM, Enzo Life Sciences), cytochalasin B (5 μM, Sigma) or antibodies directed against CD16, CD32 or CD64 (all used at 10 μg/mL, BioLegend) for 30 minutes before addition of stimuli. In some experiments, R848, at a concentration of 2 μg/mL, or neutrophil supernatant, was added 30 and 90 minutes before addition of the phagocytic stimuli, respectively.

RNA Degradation Analysis

SmRNP, labeled with Sytox Green (8 μM), was incubated in presence of huRNase (0.5 mM), IVIG, anti-RNA IgG, anti-RNP SLE IgG or a pool of SLE IgG (all at 10 μg/mL) and analyzed for RNA degradation every minute for 30 minutes at 37° C. using the Synergy 2 plate reader (BioTek). Results were normalized to the Sytox Green fluorescence level before addition of enzymes and expressed as percentage remaining RNA signal.

Neutrophil Activation

Neutrophils were activated with LPS (1 μg/mL), R848 (2.5 μg/mL), PAM3CSK4 (5 μg/mL), CpG DNA (2 μg/mL, all from Invivogen) or RNP-ICs (10 μg/mL) for 4 hours, with or without prior addition of CMK (25 μM, Enzo Life Sciences) for 60 minutes. Activation was analyzed by flow cytometry (BD FacsCanto, BD Biosciences) by assessing cell surface levels of CD66b and CD11b (BioLegend). Data was analyzed by FlowJo (Tree Star Inc).

FcgRIIA Shedding—Flow Cytometry

Neutrophils were activated by LPS (1 μg/mL), R848 (2 μg/mL), PAM3CSK4 (5 μg/mL), Loxoribine (0.1 mM), CL075 (2.5 μg/mL) or CpG DNA (2 μg/mL) for 30 minutes, followed by analysis of cell surface expression of CD32A (IV.3; Stemcell Technologies, FUN-2, BioLegend), CD16 (clone 3G8), CD64 (clone 10.1), and CD66b (all from BioLegend) by flow cytometry. For intracellular staining, neutrophils were fixed in 2% paraformaldehyde for 10 minutes, permeabilized with saponin (diluted 1:1000 in PBS) for 15 minutes and stained with anti-CD32A antibodies diluted 1:100. In some experiments, neutrophils were incubated with inhibitors (DPI (25 μM, Sigma), apocynin (100 μM, Sigma), GM-6001 (10 μM, Enzo Life Sciences), LY294002 (10 μM), cOmplete Protease Inhibitor Cocktail Tablets (1× dissolved in H₂O, Roche), neutrophil elastase IV inhibitor (25 μM, Calbiochem), E-64 (1 μM, Sigma), 4-(2-Aminoethyl)benzenesulfonyl fluoride hydrochloride (AEBSF, 0.1 mM, Sigma), CMK (25 μM, Enzo Life Sciences), cytochalasin B (5 μM, Sigma) or chymostatin (10 μg/mL, Sigma)) or recombinant furin (100 ng/mL, maximal dose tolerated by the neutrophils, Peprotech) 30 minutes prior to addition of stimuli. In some experiments cell surface levels of B cell activating factor (BAFF, Biolegend) was analyzed according to the same protocol as described above. Monocytes and pDCs were detected using antibodies to CD14 (BioLegend) and CD304 (Miltenyi Biotech), respectively.

FcgRIIA Shedding—Fluorimetry

For detection of shed FcgRIIA, neutrophils were pre-labeled with FITC-conjugated anti-CD32A antibody IV.3 (Stemcell Technologies) or FITC-conjugated anti-CD32A antibody FUN-2 (Biolegend), and washed extensively prior to activation with R848. Cell free supernatant was analyzed for shed FcgRIIA-anti-CD32A-FITC complexes by flourimetry (Synergy 2, BioTek) using anti-CD32A antibodies as a standard curve. In some experiments, cells were pre-incubated with the pan protease inhibitor cocktail (Roche).

FcgRIIA Shedding—Western Blot

Recombinant FcgRIIA (Novoprotein), biotinylated (Thermo Scientific) or non-biotinylated was incubated with neutrophil supernatant for 2 hours and analyzed for cleavage fragments using Western blot, probing with streptavidin-HRP or antibody clone IV.3, respectively.

Mass Spectrometry and Bioinformatics

Neutrophils, 4×10⁶ cells distributed in 8 tubes, were treated with medium (baseline), RNP-ICs or R848 (5 μg/mL) for 15 minutes at 37° C. Pelleted cells were lysed with 6 M Urea in 50 mM NH₄HCO₃ (Fisher Scientific) supplemented with Halt Phosphatase Inhibitor Cocktail (Thermo Scientific). Cell debris was removed by centrifugation (20,000 g for 15 minutes). For reduction and denaturation of the peptides, the samples were incubated with TCEP (37° C., 5 mM, Thermo Scientific), iodoacetamine (30 mM final concentration, BioRad) and DTT (30 mM final concentration, BioRad) for an hour each. Samples were aliquoted at 100 μL and 800 μl 25 mM NH₄HCO₃ and 200 μl MeOH (Fischer Scientific) was added to each tube followed by trypsin digestion (Promega, 1:50 w/w) for 16 hours at 37° C. Trypsinated samples were washed three times in H₂O followed by speedvac, and resuspended in 200 μL acetonitrile (ACN) with 0.1% trifluoroacetic acid (TFA, Pierce). Samples were desalted with MacroSpin Columns (The Nest Group), saturated with 80% ACN in 0.1% TFA and equilibrated with 5% ACN in 0.1% TFA. The samples were run through the columns twice and desalted samples eluted with 80% ACN in 0.1% TFA. Phosphopeptides were isolated using the TiO₂ Phosphopeptide Enrichment and Clean-up kit according to the manufacturer's instructions (Pierce). Briefly, samples were added to phosphopeptide-binding TiO₂ spin tips followed by removal of non-phosphopeptides by wash steps. Eluted phosphopeptides were cleaned in graphite columns and eluted in 50% ACN in 0.1% formic acid, followed by speedvac, and adjustment of samples to 0.1% formic acid in 5% ACN. Isolated phosphoproteins were analyzed by OrbiTrap Fusion Tribrid Mass spectrometer (Thermo Scientific). Data were extracted using MaxQuant software. Samples were normalized through dividing with the total phosphorylation level in each sample, followed by log 2 transformation. KEGG analysis was done using DAVID, and the heat map using Gene Cluster 3.0 and Java Treeview.

p47 Phox Western Blot

Neutrophils (5×10⁶ cells in 250 μL) were incubated with inhibitor of PI3K (LY294002, 10 μM) or pan protease inhibitor cocktail (lx) 30 minutes prior to addition of stimuli, and incubated for an additional 60 minutes. Neutrophil cell lysates were run on an SDS-PAGE. For the Western blot, antibodies to phosphorylated S345 (Assaybiotech) or total p47-phow (ThermoScientific) were added at 1/1000, and detected using anti-rabbit-IgG-HRP (GE Healthcare, 1/5000) followed by Super Signal West Pico Chemiluminescent Substrate (ThermoScientific) according to manufacturer's recommendations.

ROS Analysis

Neutrophils were incubated with inhibitors (LY294002 (10 μM), CMK (25 μM), DPI (25 μM) or pan protease inhibitor cocktail (lx)) for 30 minutes prior to addition of R848 (2 μg/mL) for an additional 60 minutes. DHR123 (30 μM, Sigma), was added during the last 30 minutes of incubation, and ROS analyzed by flow cytometry. For determination of extracellular ROS production upon neutrophil activation, OxyBURST® Green H2HFF BSA (25 μg/mL) was used according to the manufacturer's instructions (ThermoScientific).

Analysis of S6 and Akt Phosphorylation by Flow Cytometry

Neutrophils were activated by R848 for 15 minutes, fixed and permeabilized according to manufacturer's instructions (BioLegend), and incubated with a specific antibody recognizing phosphorylated S235/236 in S6 (Cell Signaling) or phosphorylated S473 in Akt (BD Biosciences) for 60 minutes. pS6 and pAkt levels were analyzed by flow cytometry and expressed as percent positive cells as compared to non-stimulated cells.

Incubation of PBMCs with Neutrophils or Neutrophil Supernatant

Neutrophils and PBMCs were incubated at a 2:1 ratio (500,000 vs 250,000 cells) with the pan-protease inhibitor (1×) for 30 minutes followed by R848 (2 μg/mL) for an additional 60 minutes and analyzed for FcgR levels by flow cytometry. Plasmacytoid dendritic cells were identified based on their expression of BDCA-4 (Miltenyi Biotech) and monocytes based on their expression of CD14 (Biolegend). In some experiments neutrophil supernatant (generated by incubating neutrophils with R848 for 90 minutes) were added to PBMCs with or without presence of the pan-protease inhibitor (1×), and expression of FcgRs and phagocytic ability analyzed in monocytes by flow cytometry as described above.

C5a Generation

PBMCs were incubated with or without neutrophil supernatant for 90 minutes as described above, and allowed to engulf RNP-ICs for 30 minutes. Cell-free ICs were isolated and incubated with 1% normal human serum for 3 hours at 37° C. C5a generation, as well as C5a levels in serum from healthy controls and SLE patients, was analyzed by ELISA according to the manufacturer's instructions (R&D Systems).

Statistics

For group comparisons, student's 2-tailed unpaired or paired t test was used. For the comparison between SLE patients and healthy controls the Mann-Whitney U test was used. Spearman's correlation test was used for all correlation analyses. Data were presented as bar graphs with mean and standard error of mean (SEM), or dot plots with medians. All analyses were considered statistically significant at p<0.05.

### Example 2

This example describes the development of two assay formats, IC-FLOW and NET-ELISA, which individually or combined can detect and characterize autoimmune or inflammatory diseases.

### Summary

The first assay format, referred to as IC-FLOW, relies on assessing the presence of inflammatory ICs in a sample derived from a subject by assessing by flow cytometry or similar technique the availability/presence of FcgRs on target cells or particles combined into the sample. Upon binding with ICs, the FcgR will be blocked (e.g., as presented on a particle) or internalized (e.g., as presented on a cell), and no longer available for binding to fluorescently labeled antibodies. Thus, the assay addresses availability of FcgRs, and thus the presence of ICs in the sample, by staining FcgRs with specific antibodies targeting the immune complex-binding area of the receptor and quantifying the staining by flow cytometry. The technique can be adapted to any FcgR as well as any particle and/or cell substrate that expresses at least the extracellular domain of the FcgR. We have focused on neutrophils as the FcgRIIA-bearing cell, as well as antibody IV.3 and FUN-2 for detection of available FcgRIIA on the neutrophil cell surface. In a brief description of one embodiment, patient serum is incubated with isolated neutrophils to allow for IC binding. Subsequently, antibodies towards FcgRIIA are added and available FcgRIIA determined by flow cytometry. Heat-aggregated IgG immune complexes can be used as a standard curve to estimate amounts of circulating immune complexes in patient blood.

The second assay format, referred to as NET-ELISA, relies on assessing levels of NETs, e.g., MPO-DNA, NE-DNA, or citrullinated histone-DNA complexes within biological fluids, including serum, plasma, synovial fluids, bronchoalveolar lavage, etc., using an ELISA format. Purified NETs, isolated from PMA-activated neutrophils are used as a standard curve to estimate levels of NETs in specimen.

### Results and Discussion

IC-FLOW

IC-FLOW relies on detection of FcgRs on provided target cells or particles, determining changes in FcgR availability as a measure of binding of ICs. As depicted in FIG. 2A and FIG. 2C, in absence of ICs in a sample, the availability of FcgRIIA is high and detection antibodies will be able to bind to the receptor. Two exemplary antibodies, FUN-2 and IV.3, have been used and can be implemented individually as detection antibodies or together as a combination to increase the sensitivity of the signal. As shown in FIG. 2B, upon binding of ICs, FcgR availability is reduced and FcgRIIA no longer can be stained with the antibodies, rendering less signal in the flow cytometer (FIG. 2C). The loss of FcgRIIA availability is dose-dependent (FIG. 2D), and thus useful to quantify levels of ICs in patient specimens.

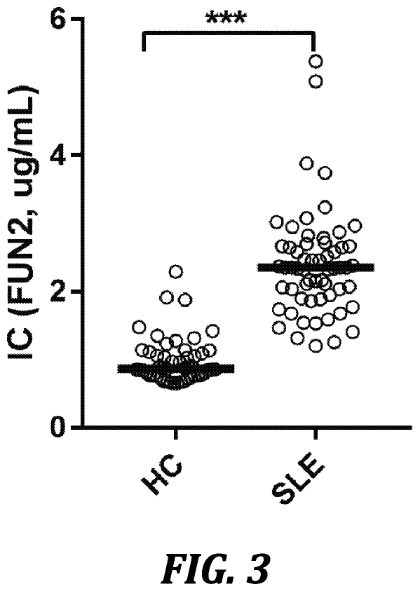

Comparing levels of ICs in serum from healthy individuals (n=50) and SLE patients (n=59), we found highly elevated levels of ICs in SLE patients (p<0.0001, FIG. 3). Screening several cohorts of patients with autoimmune and/or rheumatic disease we found that both adult and juvenile lupus patients had a high frequency of ICs (Table 2). Some ICs were also found in patients with juvenile dermatomyositis (JDM) and RA, whereas it was absent in gout patients (Table 2). Thus, though not specific for SLE, we did not find as high frequency of IC positive patients in disease controls.

TABLE 2
Frequency of IC positivity in patient cohorts

| Diagnosis | IV.3-IC | FUN2-IC | IV.3 + FUN2 |
|---|---|---|---|
| Healthy | 9/137 (7%) | 7/137 (5%) | 0/137 (0%) |
| Gout | 0/42 (0%) | 0/42 (0%) | 0/42 (0%) |
| Polymyositis | 1/7 (14%) | 1/7 (14%) | 1/7 (14%) |
| RNP + myositis | 3/12 (25%) | 1/12 (8%) | 1/12 (8%) |
| JDM | 14/50 (28%)*** | 8/50 (16%)* | 7/50 (14%)*** |
| RA | 50/351 (14%)* | 57/351 (16%)*** | 49/351 (14%)*** |
| SLE | 40/54 (74%)*** | 28/54 (52%)*** | 28/54 (52%)*** |

The cut-off for positivity was determined using the 95^{th} percentile of the healthy controls.

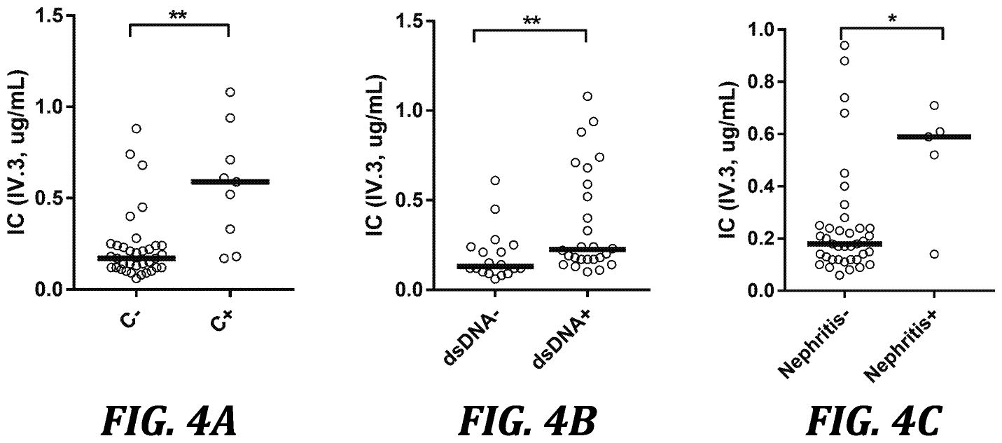

Asking whether IC-FLOW was able to detect SLE patients with active disease, we assessed the association between select disease manifestations and IC levels. As illustrated in FIGS. 4A-4C, levels of ICs were markedly associated with complement consumption, anti-dsDNA antibodies as well as presence of lupus nephritis, all of which are known to be related to IC-driven disease. Importantly, using a modified disease activity index (modSLEDAI), assessing only the clinical disease parameters, IC-FLOW, but not “gold standard” serological markers used in routine labs, were able to determine patients with active disease (modSLEDAI>4, Table 3).

TABLE 3
IC-FLOW is associated with disease activity
as compared to gold standard serological markers.

| Marker | OR | P-value |
|---|---|---|
| Anti-dsDNA antibody | 1.8 (0.5-6.5) | 0.40 |
| Low Complement C3/C4 | 2.6 (0.6-11.7) | 0.21 |
| IC-FLOW (IV.3) | 6.6 (1.6-27.2) | 0.009 |

Given that ICs are thought to initiate disease flare through complement-mediated recruitment of immune cells and FcgR-mediated tissue destruction, we next assessed whether IC-FLOW could predict upcoming flare. To investigate this we used a unique SLE cohort of 60 patients in remission whereof 40 patients would flare within three months, whereas the other 20 patients would remain in remission. Analyzing the IC levels at baseline (e.g. in remission) we found that IC levels could predict flare (Table 4). Thus, in SLE, we find highly elevated levels of ICs in the circulation, associated with, and able to predict disease flare. This would have significant clinical value in monitoring of disease activity (in particular severe nephritis), making decisions on treatment (targeting B cells particularly in these patients), as well as preventative treatment, potentially reducing the risk of flaring in nephritis, increasing quality of life (and life expectancy), as well as reducing healthcare cost avoiding expensive dialysis. Given the heterogeneity of SLE, as well as RA, it is important from a clinical perspective to understand the underlying mechanisms driving the disease. In some patients, the main contributor will be inflammatory cytokines, and in some individuals, immune complexes will be prevalent and contribute to disease. IC-FLOW, can enable clinicians to identify patients with circulating immune complexes, informing on potential treatment strategies specifically targeting this pathway, e.g. B cell depletion and/or downstream signaling pathways involved in IC-mediated inflammation, including btk pathway. This can enable personalized treatment, and avoid expensive and inadequate treatment, and subsequent side effects in patients not having evidence of IC-mediated disease. Further, IC-FLOW can be used to monitor patients during treatment to determine if they respond or not, allowing for changes in treatment strategy at an early time-point.

TABLE 4
IC-FLOW can predict disease flare in SLE

| Manifestation | OR | P-value |
|---|---|---|
| Flare | 1.2 (1.0-1.4) | 0.048 |
| Arthritis | 0.7 (0.5-0.9) | 0.02 |
| Nephritis | 1.3 (1.0-1.7) | 0.03 |

Results are presented as OR per 1 ug/mL increase in IC levels

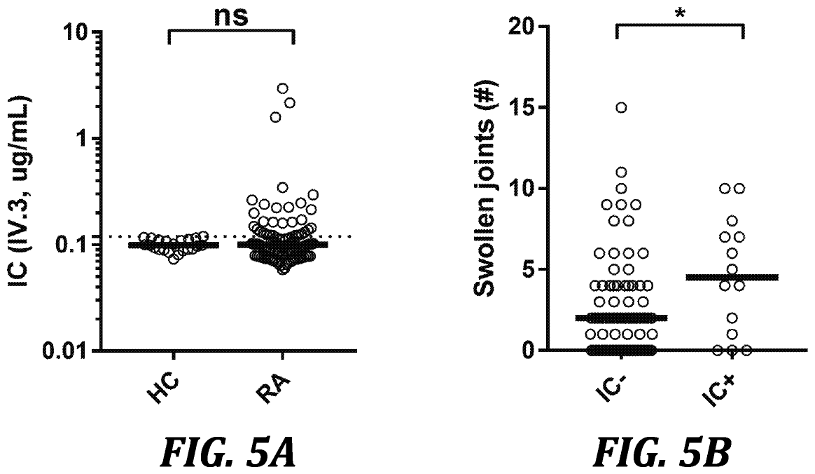

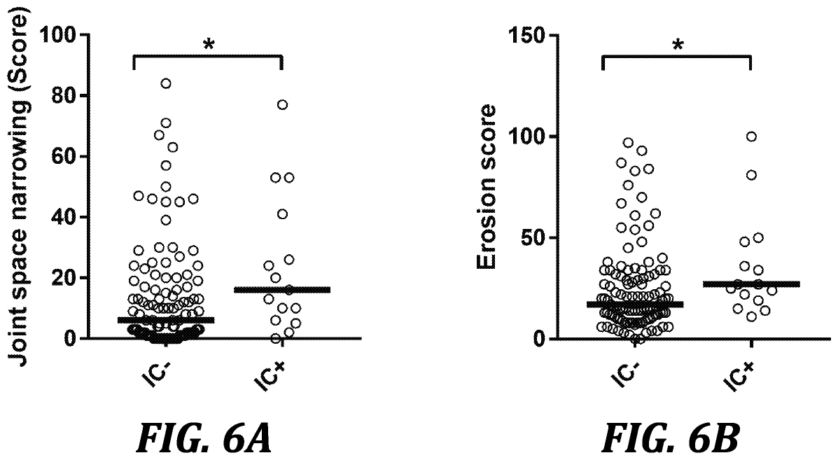

IC-FLOW can have clinical utility not only in SLE but also in other autoimmune and rheumatic diseases. To investigate this, we analyzed a large cohort of RA patients. Though, overall, RA patients did not have elevated levels of ICs, a substantial subgroup of patients (25%) had ICs (FIG. 5A). These patients also had more active disease as determined by amount of swollen joints (FIG. 5B). As per above, these patients, with a signature of IC-mediated disease, would likely benefit from B cell-targeted therapy. Considering an IC signature being related to joint inflammation, we asked if IC levels could be a predictor of disease progression, in particular development of erosive disease. To investigate this, we assessed levels of ICs at baseline in a RA inception cohort (n=250). All patients with evidence of erosive disease at baseline were removed from the analysis. The patients were followed for a mean of 8 years and subsequently assessed for disease progression. As per FIGS. 6A and 6BB, RA patients with baseline elevated levels of ICs had an increased joint space narrowing as well as erosion score, demonstrating that early detection of IC levels in RA can have predictive value in determining patients at risk of developing disabling erosive joint disease. In all, also in RA, IC-FLOW can add clinical value in identifying patients with ongoing IC-mediated disease, related to disease activity and a propensity of developing severe disabling erosive disease.

NET-ELISA

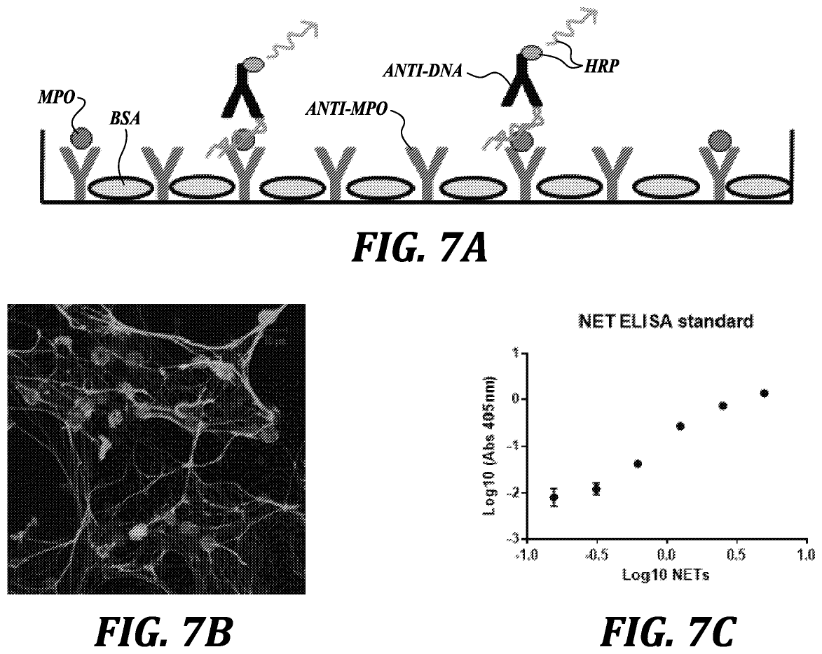

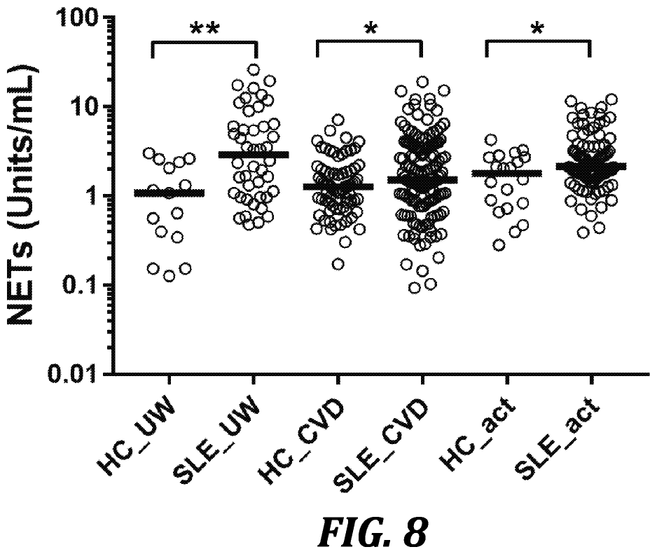

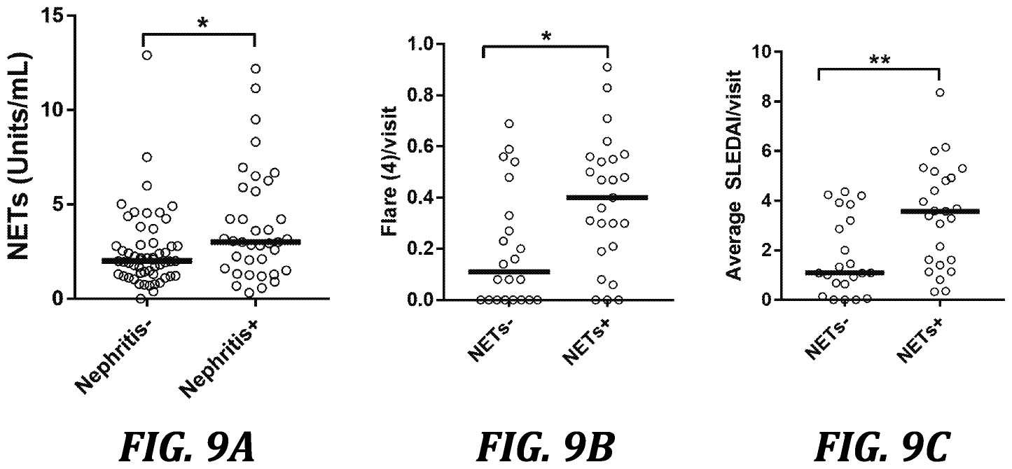

Another assay, NET-ELISA, is directed to assessing levels of circulating (NET) complexes in solution. As proof of concept, this was achieved using an ELISA assay capturing MPO (one of several protein components of NETs) and detecting dsDNA using an HRP-conjugated antibody (FIGS. 7A-7C). Specifically, anti-human MPO antibody (Biorad, #0400-0002), and HRP-conjugated anti-dsDNA antibody (Roche, #11544675001) were used as capture and detection antibody, respectively. SLE patients from three distinct cohorts all had elevated levels of NETs as compared to healthy individuals (FIG. 8). Though levels of NETs were not associated with disease activity at time-point of blood draw in SLE patients, it reflected a severe disease phenotype with increased propensity of disease flare, history of nephritis and myocardial infarction (MI; see FIGS. 9A-9C and Table 5), indicating NETs can indicate a disease phenotype, rather than disease activity.

TABLE 5
NET-ELISA identifies a severe disease phenotype in SLE.
Patients with high NET levels were predicted to have
history of nephritis and myocardial infarction, severe
disease manifestations associated with lupus-related mortality.

| Marker | OR | P-value |
|---|---|---|
| Nephritis | 3.0 (1.2-7.8) | 0.02 |
| MI | 8.0 (1.3-47.9) | 0.02 |

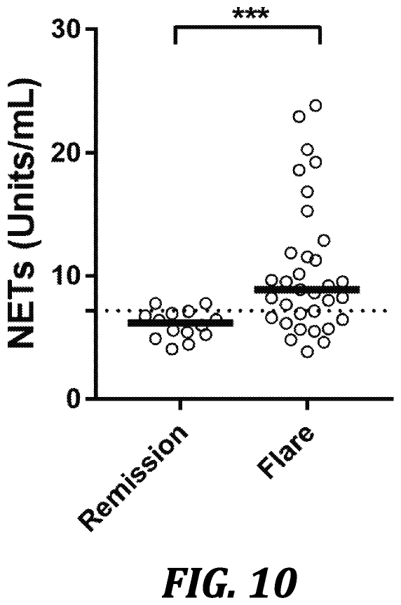

Similar to ICs inducing immune cell activation, we hypothesized that also NETs would be an early event in establishing active disease, triggering local inflammation, and triggering neutrophil-mediated organ damage. To determine whether NETs could predict lupus flare, we used samples similar as described above, e.g. 60 patients in remission. Positivity in NET-ELISA was highly associated with flare development within three months (FIG. 10; Table 6) even after adjusting for the overall increased flare frequency observed within this patient population. In all, these data indicate that NET-ELISA can be useful in identifying patients with very severe disease, requiring close monitoring, developing severe manifestations, and flaring at a high frequency. Further, NET-ELISA can provide clinicians with information on which patients are likely to flare within three months.

TABLE 6
NET-ELISA can predict disease flare in SLE. Using a
cohort of 60 SLE patients at time-point of remission,
NET-ELISA can predict which patients were to develop
a flare within three months, even after adjusting
(*) for overall flare frequency within this group.

| Variable | Manifestation | OR | P-value |
|---|---|---|---|
| NETs-high | Flare | 13.8 (2.6-73.4) | 0.002 |
| NETs (U/mL) | Flare | 1.8 (1.1-2.7) | 0.01 |
| NETs-high* | Flare | 9.5 (1.4-61.9) | 0.02 |

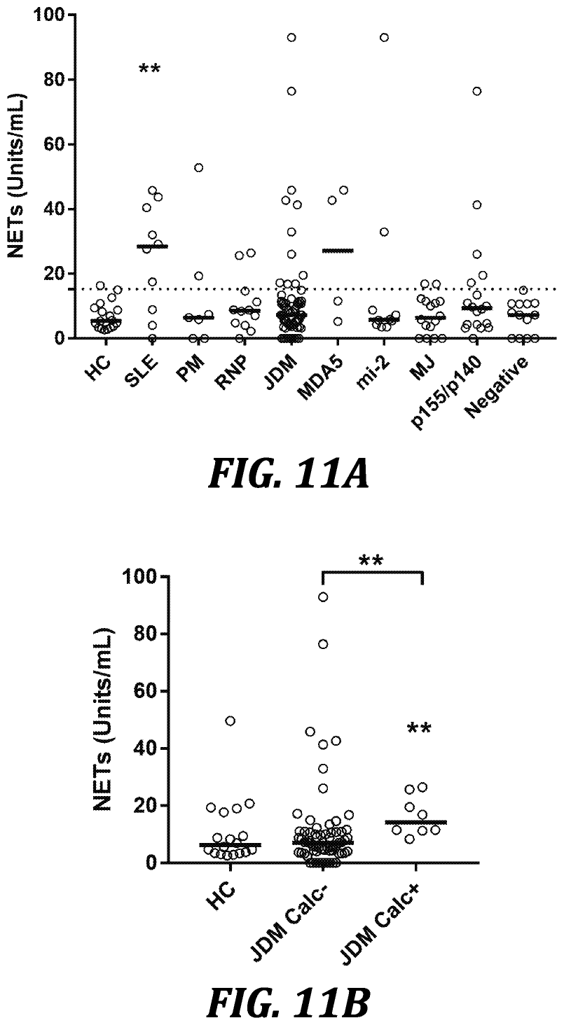

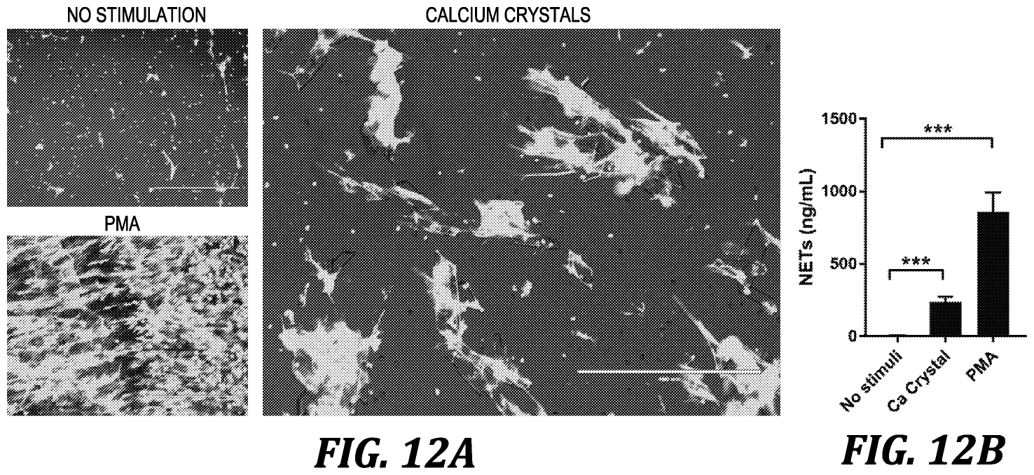

Levels of NETs were also found to be elevated in children with lupus (FIG. 11A), though not in any of the other inflammatory rheumatic conditions analyzed, including juvenile dermatomyositis (JDM). Given the spread of NET-ELISA levels, we assessed also subgroups of JDM. NETs were found to be markedly elevated in patients with calcinosis (FIG. 11B). Given this association we were able to demonstrate that calcium crystals (calcinosis) enabled neutrophils to undergo NET formation (FIGS. 12A and 12B). In all, NET-ELISA can be helpful also in JDM to identify children with calcinosis, a severe manifestation observed in these children.

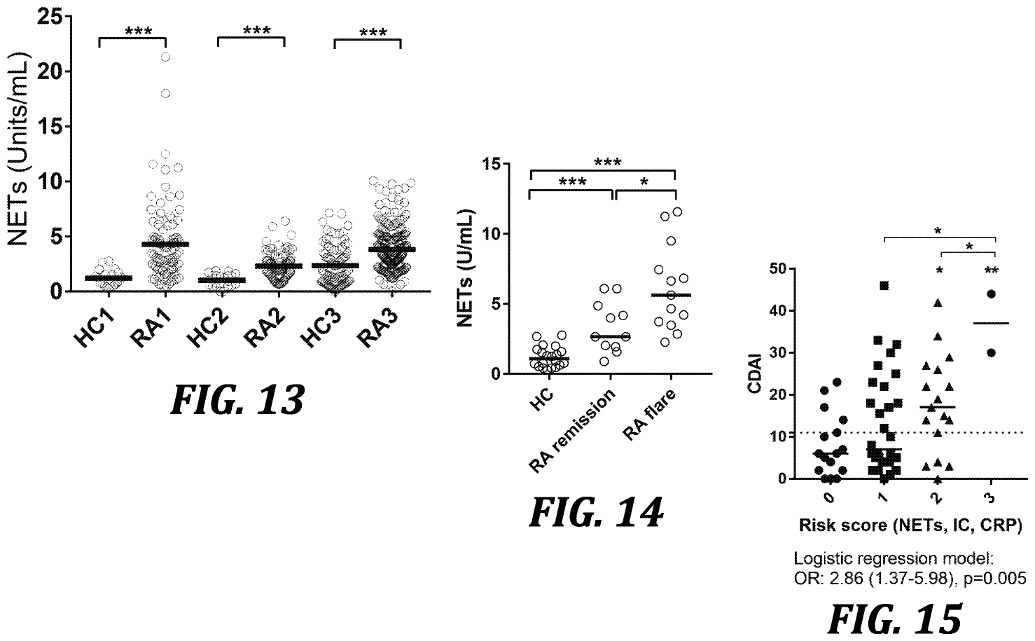

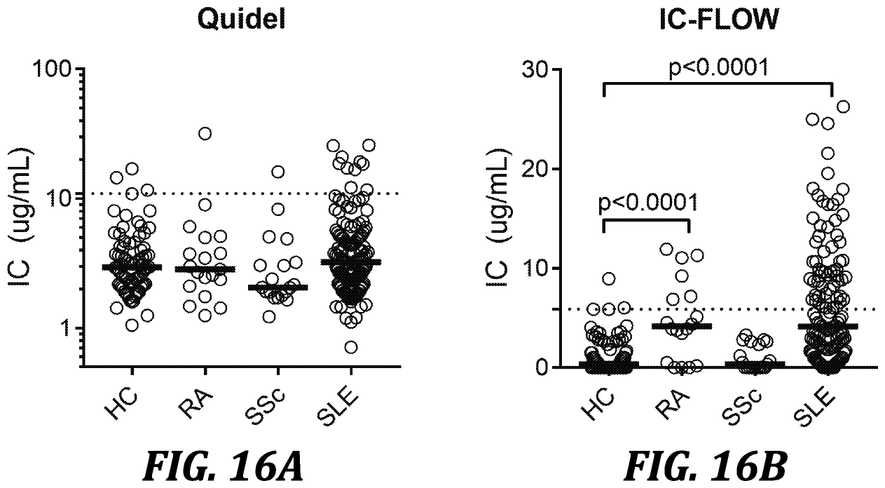

Levels of NETs were elevated in three distinct RA cohorts, whereof the third one had overall higher values due to assessment in serum (vs plasma in the other cohorts; see FIG. 13). In contrast to SLE, levels of NETs were associated with disease activity (CDAI), with NET-ELISA being a better predictor of disease activity as compared to gold standard CRP (FIG. 14; Table 7). Further, NET-ELISA can predict disease progression in RA patients, enabling clinicians to identify patients at risk of developing extra articular disease (EAD), including interstitial lung disease (ILD) and extra articular nodules, associations not observed with the currently used prognostic marker, anti-CCP (Table 8). Thus, in all, NET-ELISA is able to improve on disease activity assessment in RA while also providing prognostic insight into development of detrimental RA-associated symptoms, including extra articular disease.

TABLE 7
NET levels are associated with disease activity in RA.
Levels of NETs can better predict active disease in seropositive
RA patients as compared to gold standard CRP levels.

| Marker | OR | P-value | Sens. | Spec. |
|---|---|---|---|---|
| NETs | 6.6 (1.2-36.1) | <0.05 | 68.6 | 75.0 |
| CRP | 3.6 (0.4-32.9) | 0.40 | 37.8 | 100 |

TABLE 8
NET-ELISA can predict disease progression in RA.

| | Anti-CCP | Anti-CCP | NETs | NETs |
|---|---|---|---|---|
| Manifestation | (OR) | (p-value) | (OR) | (p-value) |
| Erosion | 6.0 (1.9-18.6) | 0.002 | 1.5 (0.7-3.5) | 0.30 |
| EAD | 2.2 (0.7-6.3) | 0.16 | 3.0 (1.2-7.6) | 0.02 |

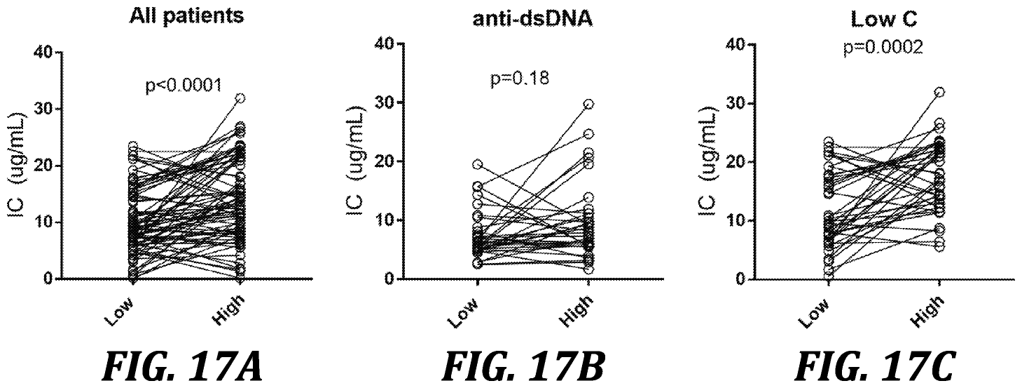

As described above, detection of IC and NET have been assessed separately as biomarkers representing a pathway of IC-mediated neutrophil activation. Combining the two markers, either alone or together with other existing biomarkers can have an enhanced effect to promote the sensitivity and specificity of the assays. In an effort to explore this, we assessed the enhanced value in combining NET-ELISA, IC-FLOW and CRP in determining RA disease activity. As shown in FIG. 15, the biomarker risk score is associated with disease activity, with the likelihood of having moderate/high disease activity increasing for every added biomarker. Thus, in RA, a combined biomarker score, including IC-FLOW and NET-ELISA can have advantages in identifying patients in flare and/or remission.

In SLE, the combined IC-FLOW and NET-ELISA assays were superior in predicting upcoming flare as compared to the individual assays, as depicted in Table 9. Finally, as shown in Table 10, the combination of IC-FLOW and NET-ELISA added significant clinical value in determining which JDM children had ongoing calcinosis as well as a history of calcinosis. This can provide a better marker to identify children with calcinosis and avoid diagnostic biopsies in these children, as well as potentially identify children earlier in their disease progression allowing for preventive treatment.

TABLE 9
the combined value of NET-ELISA
and IC-FLOW in SLE flare prediction.

| Marker | OR | P-value | Sens | Spec | AUC |
|---|---|---|---|---|---|
| IC-FLOW | 5.6 (1.1-27.8) | 0.03 | 42.9% | 88.2% | 0.66 |
| NET-ELISA | 9.0 (2.2-36.4) | 0.002 | 65.9% | 82.4% | 0.74 |
| Risk score (0/1)¹ | 11.9 (3.1-45.6) | 0.0003 | 78.6% | 76.5% | 0.78 |

¹Risk score includes patients with either NET-ELISA or IC-FLOW positivity.

TABLE 10
the combined value of NET-ELISA and IC-FLOW in
detecting JDM calcinosis JDM.

| | | Now | | Ever |
|---|---|---|---|---|
| Marker | Now sens/spec | p-value | Ever sens/spec | p-value |
| IC-FLOW | 25.0%, 88.5% | 0.28 | 28.6%, 93.8% | 0.02 |
| NET-ELISA | 37.5%, 87.5% | 0.09 | 34.8%, 93.0% | 0.004 |
| Risk score (0/1)¹ | 62.5%, 82.0% | 0.01 | 52.4%, 89.6% | 0.0003 |

¹Risk score includes patients with either NET-ELISA or IC-FLOW positivity.

In summary, IC-FLOW and NET-ELISA present new sensitive approaches to assess distinct immunological pathways operating in autoimmunity and inflammation, associating with important clinical features, enabling predictive assessment of patients with SLE and RA. Combining the two assays, with or without additional serological markers of inflammation, has added clinical value with the biomarker risk score showing ability to better predict disease flare in lupus, disease activity in RA, and disease severity (e.g., calcinosis) in JDM. These assays will provide clinical value in management of pain, fatigue and/or other symptoms in patients where clinicians currently lack objective measures to assess the disease.

### Example 3

The example describes additional studies of the IC-FLOW and NET-ELISA assays for IC-induced inflammation, and their ability to monitor disease activity as well as stratify patients based on disease severity. Specifically, described are studies that expand on the utility of these assays in SLE patients, as well as individually compare the IC-FLOW method with a commercial assay.

The IC-FLOW assay described above was configured into a 96-well format using a plate-based flow cytometer. This facilitates larger screening of samples and reduces labor intensity, as well as reduces overall sample variation.

Screening a large cohort of healthy individuals (n=217), disease controls (n=433) and SLE patients (n=361), IC-FLOW detected positive IC levels mainly in SLE patients (61%), whereas detected IC levels were low in healthy individuals (5%) and disease controls (13%). Additionally, IC levels were primarily found in a sub-group of RA patients (Table 11). In patients with active disease, 67/83 (81%) of patients were positive in IC-FLOW. In a clinical setting, patients commonly present with active disease at a time-point of diagnosis. At time-point of active disease, IC-FLOW has a high sensitivity and specificity (80.7% and 89.7%, respectively) for SLE diagnosis with ROC value of 0.85. Even in remission, IC-FLOW has a fair sensitivity and specificity (61.4%, and 89.7%, respectively), with a ROC value of 0.7 (Table 12). Thus, the IC-FLOW assay is remarkably selective in identifying a large proportion of SLE patients, in particular those with active disease, demonstrating diagnostic utility. Importantly, markers commonly used in diagnosis, including anti-dsDNA antibodies, were only found in 14% of the patients at time-point of blood draw. Therefore, in a cross-sectional setting wherein a rheumatic disease is suspected, IC-FLOW can add substantial diagnostic value for patients with SLE.

TABLE 11
IC-FLOW positivity across several rheumatic diseases

| Diagnosis | IC-FLOW | | IC-Quidel | |
|---|---|---|---|---|
| Healthy | 11/217 | (5%) | 3/80 | (4%) |
| Gout | 0/42 | (0%) | N/A | |
| Scleroderma | 0/20 | (0%) | 1/20 | (5%) |
| RA | 56/371 | (15%) | 0/20 | (0%) |
| SLE | 215/350 | (61%) | 38/351 | (11%) |
| SLE-active | 67/83 | (80.7%) | 14/83 | (16.9%) |

TABLE 12
Sensitivity and specificity of IC assays

| Diagnosis | Sens | Spec | OR | P-value | AUC |
|---|---|---|---|---|---|
| All SLE- | 61.4 | 89.7 | 13.8 (9.9-19.3) | < 0.0001 | 0.756 |
| IC FLOW | | | | | |
| All SLE- | 10.3 | 96.8 | 3.4 (1.2-9.9) | 0.02 | 0.535 |
| Quidel | | | | | |
| Active SLE^{a}- | 80.7 | 89.7 | 36.4 (19.9-66.4) | < 0.0001 | 0.852 |
| IC FLOW | | | | | |
| Active SLE- | 16.9 | 96.8 | 6.1 (1.9-19.2) | 0.002 | 0.568 |
| Quidel | | | | | |

^{a}Active disease as determined by SLEDAI > 5.

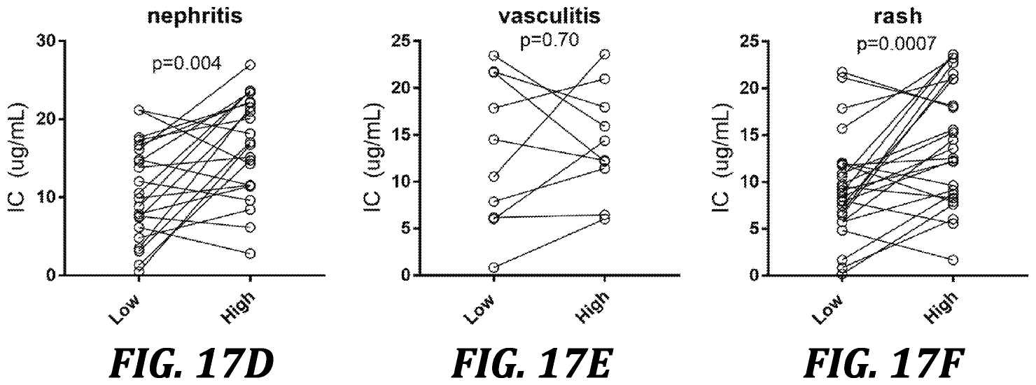

Commercially available kits for measuring IC levels commonly rely on C1q binding to circulating ICs. To determine how the IC-FLOW assay compared to C1q binding assays, IC-FLOW was compared to a commercially available IC assay from Quidel (San Diego, Calif.). As depicted in FIGS. 16A and 16B, as well as Tables 11-13, IC-FLOW performed better compared to the commercial kit, and was able to demonstrate larger differences between healthy individuals and SLE patients. Whereas IC-FLOW displayed high sensitivity and specificity across both inactive and active patients, the commercial kit had very low sensitivity (10-17%), whereas the specificity was high (97%). As such, the disclosed IC-FLOW assay, which detects the availability of FcgRIIA receptor within a sample, was superior in identifying SLE patients as compared to commercially available assay based on the C1q marker.

TABLE 13
IC-FLOW performs better than commercial assay

| Comparison^{a} | IC-FLOW | IC-Quidel |
|---|---|---|
| HC vs SLE | 11.84, p < 0.0001 | 1.10, p = 0.28 |
| HC vs SLE low | 26.03, p < 0.0001 | 1.64, p < 0.0001 |
| HC vs SLE high | 38.86, p < 0.0001 | 1.92, p < 0.0001 |
| SLE low vs SLE high | 1.49, p < 0.0001 | 1.17, p = 0.02 |

^{a}Data are represented as fold change in median IC levels.

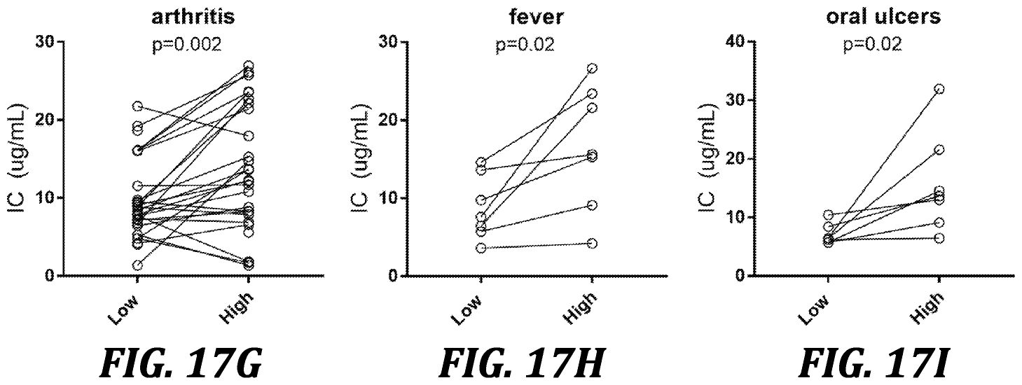

We next investigated whether the IC-FLOW associated with clinical parameter. The IC-FLOW assay was associated with complement consumption and induction of type I interferons (Table 14), both of which are indicative of IC-driven disease. Investigating individual disease activity items, IC-FLOW was elevated in several conditions, including lupus nephritis, suggesting that increased IC levels, as detected by IC-FLOW, may have broad utility in monitoring of disease activity in SLE (Table 15, and FIGS. 17A-17I). IC-FLOW could distinguish patients in remission from those having active disease (OR=3.3 (1.6-6.7), p=0.001), which was not observed for the commercial assay (OR=0.8 (0.2-2.8), p=0.70). Thus, IC-FLOW is clearly superior to the commercial assay in monitoring disease activity.

TABLE 14
Correlations between IC levels and markers of disease

| Manifestation | IC-FLOW | | IC-Quidel | |
|---|---|---|---|---|
| IC-Quidel | 0.29, | p = 0.001 | N/A | |
| SLEDAI | 0.26, | p = 0.002 | 0.18, | p = 0.04 |
| Complement C4 | −0.39, | p < 0.0001 | −0.22, | p = 0.009 |
| Complement C3 | −0.39, | p < 0.0001 | −0.30, | p < 0.0001 |
| C3dg | 0.53, | p < 0.0001 | 0.17, | p = 0.06 |
| Serum IFN I | 0.25, | p = 0.003 | 0.13, | p = 0.14 |
| PBMC IFN I | 0.26, | p = 0.002 | 0.15, | p = 0.09 |

TABLE 15
Levels of ICs are elevated in active disease

| Manifestation | IC-FLOW | IC-Quidel |
|---|---|---|
| Anti-dsDNA | P = 0.001 | P = 0.03 |
| Alopecia | P = 0.02 | P = 0.28 |
| Leukopenia | P = 0.003 | P = 0.24 |
| Low complement | P < 0.0001 | P = 0.07 |
| Lupus nephritis | P = 0.02 | P = 0.01 |
| SLEDAI > 0 | P < 0.0001 | P = 0.20 |

Next we investigated whether IC-FLOW added value to the existing “gold standard” markers of disease activity, e.g., complement consumption and anti-dsDNA antibodies. Given the incorporation of both complement and anti-dsDNA in the traditional disease activity score (SLEDAI), SLEDAI was modified to only reflect clinical disease activity (modSLEDAI). Whereas IC-FLOW could only detect high disease activity (modSLEDAI>5), anti-dsDNA antibodies could distinguish both low disease activity (modSLEDAI>0) and high disease activity (modSLEDAI>5) as illustrated in Table 16. The combination of IC-FLOW and anti-dsDNA further improved on the capacity to correctly identify patients with current active disease, suggesting additive effect of IC-FLOW in monitoring of disease activity in SLE.

TABLE 16
Combining IC-FLOW with serological markers of disease activity

| ModSLEDAI^{a} | >0 | | >5 | |
|---|---|---|---|---|
| IC-FLOW | 1.9 (0.8-4.3) | p = 0.14 | 3.8 (1.1-13.2) | p = 0.04 |
| Anti-dsDNA | 3.5 (1.8-7.1) | p < 0.0001 | 3.9 (2.0-7.5) | p < 0.0001 |
| Low | 1.5 (0.8-2.8) | p = 0.16 | 2.4 (1.2-4.5) | p = 0.009 |
| complement | | | | |
| IC-FLOW + | 5.9 (2.5-13.6) | p < 0.0001 | 4.3 (2.1-8.9) | p < 0.0001 |
| dsDNA | | | | |
| IC-FLOW + | 1.6 (0.9-3.1) | p = 0.13 | 2.6 (1.3-5.1) | p = 0.007 |
| Low C | | | | |
| dsDNA + | 3.5 (1.5-8.2) | p = 0.003 | 4.1 (1.9-8.5) | p < 0.0001 |
| Low C | | | | |
| All three | 2.9 (1.2-6.9) | p = 0.02 | 3.3 (1.5-7.2) | p = 0.003 |
| markers | | | | |

^{a}Modified SLEDAI was used, excluding any score from anti-dsDNA and complement consumption from the overall disease activity score.

Given that IC-FLOW (as well as NET-ELISA) identified patients with severe lupus nephritis (Table 17), we next asked whether there would be benefit of combining the two biomarker assays, IC-FLOW and NET-ELISA. As illustrated in Table 18, combining IC-FLOW and NET-ELISA improved the capacity to identify patients with a severe disease progression, including lupus nephritis and cardiovascular disease. The combined biomarker panel was even better than gold standard, e.g. anti-dsDNA antibodies (OR=3.4 (1.3-9.1), p=0.01) and anti-C1q antibodies (OR=2.4 (0.9-6.4), p=0.08), in identifying patients with lupus nephritis.

TABLE 17
Levels of ICs are associated with severe lupus nephritis

| Manifestation | IC-FLOW | IC-Quidel |
|---|---|---|
| Anti-dsDNA ever | P < 0.0001 | P = 0.21 |
| Nephritis ever | P = 0.002 | P = 0.004 |

TABLE 18
Combining IC-FLOW and NET-ELISA improves
identification of severe disease

| Manifestation | IC-FLOW | NET-ELISA | Combined |
|---|---|---|---|
| Nephritis ever | 2.3 (1.1-4.8) | 2.4 (0.9-6.7) | 5.0 (1.2-20.1) |
| | p = 0.02 | p = 0.10 | p = 0.03 |
| MI ever | 2.8 (0.5-15.8) | 8.7 (1.6-47.4) | 17.9 (3.0-105.1) |
| | p = 0.24 | p = 0.01 | p = 0.001 |
| Arterial | 0.8 (0.3-2.1) | 3.7 (1.2-11.4) | 4.4 (1.1-17.0) |
| thrombosis | p = 0.64 | p = 0.02 | p = 0.04 |

In summary, this supplemental study, focusing on IC-FLOW in SLE, establishes the following main observations:
1) IC-FLOW can diagnose SLE, in particular in active disease.
2) IC-FLOW is superior to commercial assays in a) identifying SLE patients, and b) in monitoring of disease activity
3) IC-FLOW detects patients with active, and severe, disease
4) Combined with anti-dsDNA, IC-FLOW improves on monitoring of disease activity
5) Combined with NET-ELISA, IC-FLOW improves on detection of severe disease

In a clinical setting, IC-FLOW can contribute significant clinical value in improving early diagnosis of SLE patients, allowing for early preventive interventions, and reduction of long-term disabling disease. In established disease, IC-FLOW adds significant value in monitoring disease activity, as well as identifying patients with a severe disease phenotype, prone to develop lupus nephritis and cardiovascular disease. Once identified, such patients should be monitored closely and treated more aggressively to prevent development of these manifestations.

While illustrative embodiments have been illustrated and described, it will be appreciated that various changes can be made therein without departing from the spirit and scope of the invention.

1-58. (canceled)

59. A kit, comprising:
A) a capture affinity reagent that binds to a neutrophil extracellular trap (NET) at a first epitope, and a detection affinity reagent that binds to the NET at a second epitope; and/or
B) a particle expressing FcgRIIA receptor, or an extracellular domain thereof, and one or more affinity reagents that compete with IC s for binding the extracellular domain of FcgRIIA receptor expressed on the particle.

60. The kit of claim 59, wherein the capture affinity reagent is immobilized on a solid substrate.

61. The kit of claim 59, wherein the NET comprises a complex of myeloperoxidase (MPO) and nucleic acid, a complex of neutrophil elastase (NE) and nucleic acid, and/or a complex of citrullinated histones and DNA.

62. The kit of claim 61, wherein the first epitope is on the MPO, NE, or citrullinated histone on the NET complex, and the second epitope comprises double stranded DNA.

63. The kit of claim 61, wherein the first epitope comprises double stranded DNA and the second epitope is on the MPO, NE, or citrullinated histone on the NET complex.

64. The kit of claim 59, wherein the detection affinity reagent is detectably labeled.

65. The kit of claim 59, further comprising a second detection affinity reagent that specifically binds to the detection affinity reagent, wherein the second detection affinity reagent is detectably labeled.

66. The kit of claim 59, wherein the one or more affinity reagents that compete with ICs for binding an extracellular domain of FcgRIIA receptor on the particle expressing FcgRIIA receptor comprises a first affinity reagent and a second affinity reagent, wherein the first affinity reagent and the second affinity reagent each compete with ICs for binding the extracellular domain of FcgRIIA receptor but wherein the first affinity reagent and the second affinity reagent do not mutually compete for binding the extracellular domain of FcgRIIA receptor.

67. The kit of claim 59, wherein the one or more affinity reagents are detectably labeled.

68. The kit of claim 59, wherein the capture affinity reagent, the detection affinity reagent, the second detection affinity reagent, and/or the one or more affinity reagents, are independently an antibody, or a fragment or a derivative thereof.

69. The kit of claim 59, wherein the one or more affinity reagent are selected from antibody IV.3 or antibody 8.7; FUN-2; or an antigen-binding fragment or derivative thereof.

70. A method of increasing phagocytosis of nucleic acid-containing immune complexes (ICs) by neutrophils, comprising:
contacting the neutrophils with an agent that inhibits activity of TLR7, TLR8, and/or TLR9.

71-76. (canceled)

77. The method of claim 70, wherein the method is performed in vivo and comprises reducing nucleic acid-containing immune complex (IC)-driven inflammation in a subject in need thereof, wherein the agent is a TLR7-9 inhibitory deoxynucleotide (iODN) that inhibits activity of TLR7, TLR8 and/or TLR9, wherein the method comprises administering to the subject an effective amount of the TLR7-9 iODN.

78-99. (canceled)

100. A kit, comprising:
a first affinity reagent that specifically binds to a first epitope in an N-terminal domain of the FcgRIIA receptor; and
a second affinity reagent that specifically binds to a second epitope in an extracellular domain of the FcgRIIA that is not in the N-terminal domain.

101-106. (canceled)

107. The kit of claim 66, wherein the first affinity reagent is labeled with a first detectable label and the second affinity reagents is labeled with a second detectable label, and wherein the first detectable label and the second detectable label are different.

108. The kit of claim 59, wherein the particle is a neutrophil, monocyte, liposome, mixed micelle, platelet, or synthetic bead.

109. The kit of claim 59, wherein the particle is a circulating cell obtained from one or more donor individuals with no inflammatory or autoimmune disease.

110. A method of using the kit of claim 59 to detect the presence of immune complexes (ICs) in a biological sample obtained from a subject, comprising:
contacting a biological sample with one or more particles expressing FcgRIIA receptor on the surface;
contacting the biological sample with one or more affinity reagents that compete with ICs for binding an extracellular domain of FcgRIIA receptor on the one or more particles; and
detecting the binding of the one or more affinity reagents to one or more particles in the biological sample;
wherein reduced binding levels of the one or more affinity reagents compared to a reference binding level indicates the presence of elevated levels of ICs in the subject.

111. The method of claim 110, further comprising:
detecting a level of neutrophil extracellular traps (NETs) in a biological sample obtained from the subject; and
determining the status of an autoimmune or inflammatory disease in the subject, comprising, wherein a combination of a higher level of NETs compared to a NET reference level and a higher level of ICs compared to an IC reference level indicate the presence or elevated risk of an autoimmune or inflammatory disease in the subject.

112. A method of using the kit of claim 100 to detect circulating cells with a truncated FcgRIIA receptor, comprising:
contacting a sample containing one or more neutrophils and/or monocytes obtained from a subject with the first affinity reagent that specifically binds to a first epitope in an N-terminal domain of the FcgRIIA receptor and the second affinity reagent that specifically binds to a second epitope in an extracellular domain of the FcgRIIA that is not in the N-terminal domain; and
detecting the binding of the first affinity reagent and the second affinity reagent to the one or more neutrophils and/or monocytes in the sample;
wherein reduced binding levels of the first affinity reagent compared to the second affinity reagent indicate one or more neutrophils and/or monocytes with truncated FcgRIIA receptor.


SEQUENCE LISTING (9 sequences)

SEQ ID NO: 1 - protein, 316 aa, Homo sapiens
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRATDDDKNIYLTLPPNDHVNSNN

SEQ ID NO: 2 - DNA, 18 bp, Artificial sequence
  Synthetic
tgctcctggaggggttgt

SEQ ID NO: 3 - DNA, 18 bp, Artificial sequence
  Synthetic
tgctcctggaggggttgt

SEQ ID NO: 4 - DNA, 15 bp, Artificial sequence
  Synthetic
tcctggcggggaagt

SEQ ID NO: 5 - DNA, 11 bp, Artificial sequence
  Synthetic
tcctggagggg

SEQ ID NO: 6 - DNA, 11 bp, Artificial sequence
  Synthetic
tcctggagggg

SEQ ID NO: 7 - DNA, 20 bp, Artificial sequence
  Synthetic
ctcctattggggtttcctat

SEQ ID NO: 8 - DNA, 20 bp, Artificial sequence
  Synthetic
ctcctattggggtttcctat

SEQ ID NO: 9 - DNA, 18 bp, Artificial sequence
  Synthetic
cctcaatagggtgagggg